SEQUENCE LISTING (11 sequences)

SEQ ID NO: 1 - protein, 735 aa, Adeno-associated virus 2 [sequence omitted]

SEQ ID NO: 2 - protein, 598 aa, Adeno-associated virus 2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 533 aa, Adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - DNA, 4642 bp, Artificial Sequence
  source: /note="Description of Artificial Sequence: Synthetic polynucleotide"
gctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtctcgctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttccc

SEQ ID NO: 5 - DNA, 4642 bp, Artificial Sequence
  source: /note="Description of Artificial Sequence: Synthetic polynucleotide"
gctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttccc

SEQ ID NO: 6 - DNA, 4642 bp, Artificial Sequence
  source: /note="Description of Artificial Sequence: Synthetic polynucleotide"
gctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtctcgctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttccc

SEQ ID NO: 7 - DNA, 2877 bp, Artificial Sequence
  source: /note="Description of Artificial Sequence: Synthetic polynucleotide"
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctcggaactaataccctcgctacaggcagtggcgcaccactcgcagacaataacgagggcgccgacggagtgggtaattcctcgggaaattggcattgcgattccacatggctcggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - DNA, 2592 bp, Artificial Sequence
  source: /note="Description of Artificial Sequence: Synthetic polynucleotide"
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctcggaactaataccctcgctacaggcagtggcgcaccactcgcagacaataacgagggcgccgacggagtgggtaattcctcgggaaattggcattgcgattccacatggctcggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagc

SEQ ID NO: 9 - DNA, 5595 bp, Artificial Sequence
  source: /note="Description of Artificial Sequence: Synthetic polynucleotide"
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctcggaactaataccctcgctacaggcagtggcgcaccactcgcagacaataacgagggcgccgacggagtgggtaattcctcgggaaattggcattgcgattccacatggctcggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctcggaactaataccctcgctacaggcagtggcgcaccactcgcagacaataacgagggcgccgacggagtgggtaattcctcgggaaattggcattgcgattccacatggctcggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactaca

SEQ ID NO: 10 - DNA, 10010 bp, Artificial Sequence
  source: /note="Description of Artificial Sequence: Synthetic polynucleotide"
cccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatccagtcgcgctgctctcgtcgatccgctaggactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgtggttacctacaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccgtagatctctcgagcagcgctgctcgaggcaagcttctgcaggtcgactctagaggatccctacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcagaattcaatcgatgttcgaatcccaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttgtgggcctatagactctataggcggtacttacgtcactcttggcacggggaatccgcgttccaatgcaccgttcccggccgggattcgaatccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgcaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattgacgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagctccacgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgagctagcaaaaaatgcccggagcgggacttgaacccgcacagcgcgaacgccgagggatttagagtcccttgtgtctaccgattccaccatccgggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaggtacccctagcaaaaaatgcccggagcgggacttgaacccgcacagcgcgaacgccgagggatttagagtcccttgtgtctaccgattccaccatccgggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaggtacccctagcaaaaaatgcccggagcgggacttgaacccgcacagcgcgaacgccgagggatttagagtcccttgtgtctaccgattccaccatccgggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaggtacccctagcaaaaaatgcccggagcgggacttgaacccgcacagcgcgaacgccgagggatttagagtcccttgtgtctaccgattccaccatccgggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaggtacccctaggtgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccgccaccatggaagagcaataccgcccggaagagatagaatccaaagtacagcttcattgggatgagaagcgcacatttgaagtaaccgaagacgagagcaaagagaagtattactgcctgtctatcctcccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgtccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacgcgtttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgactctagaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggatctatgcgcgctacacttgcccgcagtacaaagaaggtatgctggattccgaagcggctaactactggctgccggtggatatcgctattggtggtattgaacacgccattatgggtctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggcgggcccgtttaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccctaggggtgcgagcggatcgagcagtgtcgatcactactggaccgcgagctgtgctgcgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 11 - DNA, 10183 bp, Artificial Sequence
  source: /note="Description of Artificial Sequence: Synthetic polynucleotide"
cccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatccagtcgcgctgctctcgtcgatccgctagggcggccgcaaatacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgtggttacctacaaaatcagaaggacagggaagggagcagtggttcacgcctgtaatcccagcaatttgggaggccaaggtgggtagatcacctgagattaggagttggagaccagcctggccaatatggtgaaaccccgtctctaccaaaaaaacaaaaattagctgagcctggtcatgcatgcctggaatcccaacaactcgggaggctgaggcaggagaatcgcttgaacccaggaggcggagattgcagtgagccaagattgtgccactgcactccagcttggttcccaatagaccccgcaggccctacaggttgtcttcccaacttgccccttgctccataccacccccctccaccccataatattatagaaggacacctagtcagacaaaatgatgcaacttaattttattaggacaaggctggtgggcactggagtggcaacttccagggccaggagaggcactggggaggggtcacagggatgccacccgtagatctctcgagcagcgctgctcgaggcaagcttctgcaggtcgactctagaggatccctacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccattcagaattcaatcgatgttcgaatcccaattctttgccaaagtgatgggccagcacacagaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttgtgggcctatagactctataggcggtacttacgtcactcttggcacggggaatccgcgttccaatgcaccgttcccggccgggattcgaatccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgcaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattgacgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagctccacgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggctcgatccgctcgcaccctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccaggggcgtagttcaattggtagagcaccggtcttcaaaaccgggtgttgggagttcgagtctctccgcccctgccattttttgctaggttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatgactaagcccatcgtttttgctggcgcacagccctcaggtgaattgaccattggtaactacatgggtgcgctgcgtcagtgggtaaacatgcaggatgactaccattgcatttactgtatcgttgaccaacacgcgatcaccgtgcgccaggatgcacagaagctgcgtaaagcgacgctggatacgctggccttgtatctggcttgtggtatcgatcctgagaaaagcaccatttttgttcagtcccacgtgccggaacatgcacagttaggctgggcactgaactgctatacctacttcggcgaactgagtcgcatgacgcagtttaaagataaatctgcgcgttatgccgagaacatcaacgctggtctgtttgactatccggtgctgatggcagcggacatcctgctgtatcaaactaatctgggtccttgtggtgaagaccagaaacagcacctcgaactgagccgcgatattgcccagcgtttcaacgcgctgtatggcgagatctttaaggtgccggagccgtttattccgaaatctggcgcgcgcgtaatgtcgctgctggagccgaccaagaagatgtccaagtctgacgataatcgcaataacgttatcggcctgctggaagatccgaaatcggtagtgaagaaaatcaaacgtgcggtcactgactccgacgagccgccggtagttcgctacgatgtgcagaacaaagcgggcgtttccaacctgttggatatcctttcagcggtaacgggccagagcatcccagaactggaaaaacagttcgaaggcaagatgtatggtcatctgaaaggtgaagtggctgatgccgtttccggtatgctgactgaattgcaggaacgctatcaccgtttccgcaacgatgaagccttcctgcaacaggtgatgaaagatggcgcggaaaaagccagcgcgcacgcttcccgtacgctaaaagcggtgtacgaagcgattggttttgtggcgaagccgtaagaattcaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggactaggggtgcgagcggatcgagcagtgtcgatcactactggaccgcgagctgtgctgcgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

(19) United States
(12) Patent Application Publication
(10) Pub. No.: US 2023/0175013 A1
(43) Pub. Date: Jun. 8, 2023

(54) CONTROLLED MODIFICATION OF ADENO-ASSOCIATED VIRUS (AAV) FOR ENHANCED GENE THERAPY

(71) Applicant: TRUSTEES OF BOSTON COLLEGE, Chestnut Hill, MA (US)

(72) Inventors: Abhishek CHATTERJEE, Brookline, MA (US); Rachel E. KELEMEN, Newton, MA (US); Sarah B. ERICKSON, Boston, MA (US); Quan PHAM, Chestnut Hill, MA (US)

(21) Appl. No.: 17/922,425

(22) PCT Filed: May 3, 2021
(86) PCT No.: PCT/US2021/030489

§ 371 Date: Oct. 31, 2022

Related U.S. Application Data
(60) Provisional application No. 63/018,573, filed on May 1, 2020.

(51) Int. Cl. C12N 15/86

(52) U.S. Cl. CPC C12N 15/86; A61K 48/00

(57) ABSTRACT

The present invention discloses platforms for chemically modify AAV capsids with control over site and stoichiometry. An AAV packaging system is described that allows the introduction of site-directed natural and unnatural amino acid mutations into any subset of the three capsid proteins. These engineered residues can be subsequently used to chemically functionalize the resulting capsids with precise control over site and stoichiometry. Such controlled modification strategy can be used to attach a wide variety of entities to AAV capsids to engineer its tropism, immunogenicity, etc.

### GOVERNMENT SUPPORT

This invention was made with Government support under grant number 1817893, awarded by the National Science Foundation. The Government has certain rights in the invention.

### RELATED APPLICATIONS

This application claims the benefit under 35 U.S.C. § 119(e) of U.S. Provisional Application No. 63/018,573, filed on May 1, 2020, which is incorporated herein by reference in its entirety.

### INCORPORATION BY REFERENCE OF MATERIAL IN ASCII TEXT FILE

This application incorporates by reference the Sequence Listing contained in the following ASCII text file: : 0342_0010WO1_SL.txt; created May 3, 2021, 76,879 bytes in size.

### FIELD OF THE INVENTION

The present invention is directed to the field of biotechnology, focusing on the development of engineered AAV-based gene-therapy vectors through the controlled chemical modification of the virus capsid.

### BACKGROUND OF THE INVENTION

Adeno-Associated Virus (AAV) has emerged as one of the most promising delivery vehicles for gene therapy. It is naturally replication deficient, exhibits only a low innate immune response, efficiently infects dividing as well as non-dividing cells, and provides stable, long-term expression of the delivered therapeutic gene in vivo. Indeed, several AAV-based gene therapies have recently been approved, and many others are currently in clinical trial. Despite the promise of AAV-based gene therapy to provide cures for numerous diseases that are currently untreatable, there are several key challenges. For example, nearly all of the current approaches rely on the natural serotypes of AAV, which exhibit restrictive innate tissue-tropism. Naturally, diseases associated with cell-types that are efficiently infected by these vectors can be targeted by this approach. However, many other diseases that occur in tissues that are not readily infected by existing AAV vectors currently remain refractory. The ability to reengineer the tissue-tropism of existing AAV vectors will be highly useful for creating custom vectors that could deliver genetic payloads to a desired location, substantially expanding the scope of AAV-based gene therapy. Additionally, many patients have pre-existing immunity to existing AAV vectors. Furthermore, administration of therapeutic AAV vectors elicits strong adaptive immune response, prohibiting a second dose of the same vector. The ability to engineer existing AAV vectors to evade such immune-recognition will be key for the development of sustainable gene therapies. Directed evolution has been used to develop AAV capsids with more therapeutically desirable traits. However, this approach is often unreliable for producing acceptable solutions, and the resulting mutant AAV capsids frequently show packaging deficiencies.

The ability to display activity-modulating entities on existing AAV capsids in a controlled manner provides an attractive modular approach to engineer their properties. Numerous methods have been attempted towards this goal, which include N-terminal extension of the minor capsid proteins, insertion of peptide loops in permissive sites of the capsid proteins, and modification of existing amino acids, such as lysine, on the capsid surface. However, N-terminal extension and loop insertion often disrupt the assembly of the complex AAV capsid. Because of the delicacy of the AAV capsid, insertion of peptide loops is restricted to very few locations and the resulting capsids often exhibit suboptimal packaging. These methods provide limited flexibility in optimizing the placement of the activity-modulating moiety, the structural variety of entities that can be displayed, as well as the ability to optimize the number of targeting moieties displayed on the capsid surface.

### SUMMARY OF THE INVENTION

It has been possible to produce AAV incorporating UAAs with an azido functionality, which was then conjugated to a cyclooctyne-modified cRGDFC via strain-promoted azide-alkyne cycloaddition (SPAAC), thus retargeting the virus to the αVβ3 integrin receptor that is overexpressed in certain types of cancer cells. However, incorporation of UAAs as previously described has been restricted to the incorporation of the UAA to all 60 capsid proteins of AAV. As described herein, it has been demonstrated that the over-modification of all 60 capsid proteins (including minor capsid proteins VP1 and VP2 as well as the major capsid protein VP3) of the AAV virus particle is strongly detrimental for its infectivity. The ability to control the number of amino acid mutations to produce variant capsids to incorporate/introduce a specific number of naturally-occurring and/or UAA modifications/mutations per capsid (relative to the wild-type AAV) is a key feature of the present invention to allow the synthesis of infectious genetically-modified adeno-associated viruses comprising variant capsid proteins with mutated amino acid residues. These mutated residues in the variant capsid proteins permit the attachment (e.g., covalent attachment) of biological and/or chemical entities (also referred to herein as “groups”) to introduce bioconjugation “handles” into the AAV in a site-specific manner resulting in the incorporation/introduction of a defined number of activity-modulating groups that enable optimally engineering AAV function.

The present invention encompasses genetically engineered/genetically-modified adeno-associated virus (AAV) with modification/mutations at site specific locations in one, or more, of the capsid proteins relative to the corresponding sites of the wild-type AAV, and methods of making these genetically-modified viruses. Importantly, as described herein, modifications can be limited to one, or more specific, chemo-selective location(s) in a single capsid protein such as VP1, VP2 or VP3, or multiples of these capsid proteins, such as in both VP1 and VP2, without affecting the infectivity of the AAVparticle (i.e., wherein the infectivity of the genetically-modified AAV is essentially comparable to, or slightly altered from, the infectivity of wild-type AAV under similar biological conditions).

AAV is a small non-enveloped parvovirus with a 4.5 kb single stranded DNA genome which codes for the non-structural Rep and AAP proteins and the structural Cap proteins VP1, VP2, and VP3, which share a common C terminus and differ by N-terminal extensions. Unlike enveloped viruses, which have a lipid membrane decorated with surface proteins, AAV has tightly packed icosahedral capsid consisting only of 60 copies of the proteins VP1, VP2, and VP3 in a roughly 1:1:10 stoichiometry. AAV is replication-deficient -- complete virus production and escape requires the presence of a helper virus such as adenovirus. AAV has a number of different serotypes with different natural tropisms. For example, one serotype described herein is AAVtype 2 (AAV2), which targets cells expressing the heparan sulfate proteoglycan receptor.

Adeno-associated virus has emerged as the most promising candidate for human gene therapy. This virus boasts desirable properties like low immunogenicity and long-term gene expression, ideal for a gene therapy vector. However, many of the other properties of these virus often do not align with therapeutic needs. For example, natural serotypes of the virus have distinct cell specificities, which restrict which cells/tissues can be targeted for human gene therapy. The ability to functionalize existing AAV vectors, with a precise control over site and stoichiometry offers an attractive avenue to introduce therapeutically desirable traits such as cell-specificity and immune-evasion.

Described herein is technology to precisely, and site-specifically, genetically-engineer AAV by introducing amino acids, such as UAAs, with entities or groups such as bioconjugation entities or “handles” (e.g., the natural amino acid cysteine, or unnatural amino acids with a broad range of bioconjugation chemistries) into the minor capsid proteins of AAV, The minor capsid proteins VP1 and VP2 are present at 5 copies each per AAV capsid. As described herein, the constructs and methods of the present invention enable site-selective modification of any of the three capsid proteins, but specifically modify VP1 or VP2 or both VP1 and VP2, allowing the attachment of 5 or 10 groups per fully-assembled AAV capsid. (the term “fully-assembled” as used herein means virus assembly incorporating all three capsid proteins, the minor VP1 and VP2 capsid proteins as well as the major capsid proteins, VP3 to form an infectious virus particle). The present method further allows the incorporation of bioconjugation handles (up to two bioconjugation handles per capsid protein) enabling precise attachment of 15 or 20 bioconjugation groups per capsid.

More specifically, the present invention encompasses a method to produce AAV vectors, in which any one of, or combination of, the minor capsid proteins VP1 and VP2, can be site-specifically chemically modified. Since the three capsid proteins are present at different stoichiometry (5, 5, and 50 copies of VP 1, VP2 and VP3 respectively per capsid), this method provides the ability to create homogeneous AAV conjugates where modifications can be introduced into capsids with precise control over both the site and the number of modifications and retain biological activity of the virus, especially its targeted cellular infectivity.

Using engineered aminoacyl-tRNA synthetase (aaRS)/tRNA pairs that suppress a nonsense codon, as described herein, it is possible to site-specifically incorporate natural and unnatural amino acids (UAAs) into the capsid proteins. Due to their small size, UAAs are well-tolerated in the AAV capsid at many positions.

Encompassed by the present invention is a genetically-modified adeno-associated virus (AAV) wherein the AAV capsid comprises at least one variant minor capsid protein VP1, VP2, or both VP1 and VP2, wherein the variant minor capsid protein is mutated at one, or more, amino acid residue sites to incorporate a natural amino acid not present in the wild-type AAV, or an unnatural amino acid (UAA) relative to the wild-type AAV VP1 or VP2 capsid protein. Importantly, the genetically-modified AAV s described herein retain their infectivity as compared to the wild-type AAVunder comparable conditions.

In particular, the present invention encompasses a genetically-modified adeno-associated virus (AAV) comprising a variant minor capsid protein, wherein the capsid coding sequence (SEQ ID NO: 1) is mutated at the translation origin of the AAV VP1 or VP2 or VP3 capsid protein open reading frame (ORF) to prevent translation of VP1, VP2 or both VP 1 and VP2, resulting in an AAV capsid protein with VP1, VP2 , VP3 or both VP1 and VP2 deleted. The deleted/missing capsid protein is then provided (i.e, expressed) in trans from a second capsid coding sequence encoding the deleted minor capsid protein(s) resulting in a genetically-modified AAV with one, or more variant capsid proteins. VP2 is also represented by SEQ ID NO:2 and VP3 is also represented by SEQ ID NO:3 herein.

The genetically-modified AAV capsid protein comprises SEQ ID NO: 1, or a sequence comprising at least about 70%, 75%, 80%, 85%, 90%, 95% , 99%, or between 90% and 99%, sequence identity of SEQ ID NO: 1. The variant capsid protein is mutated at specific sites to delete VP 1, VP2 or both VPP1 and VP2 (e.g., as shown in FIG. 2). For example, at position T454 of SEQ ID NO: 1 a stop codon can be inserted using the techniques described herein. Such techniques are also described in, for example, PCT/US2020/055834; PCT/US2020/038766 or PCT/US2020/029567, the teachings of which are incorporated herein in their entirety, by reference. More specifically, the stop codon incorporated in the variant capsid protein can be a TAG, TAA or TGA suppressor stop codon.

Specifically encompassed by the present invention is the minor capsid protein CMV-VP1-delVP23 (FIG. 20, SEQ ID NO: 7)); CMV-VP2-delVP3 (FIG. 21, SEQ ID NO:9) and CMV-VP1-VP2-delVP3 (FIG. 22, SEQ ID NO: 10). As described herein, the CMV promoter sequence is illustrated but any suitable promoter sequence can be incorporated. Additionally, the minor capsid proteins can incorporate naturally-occurring or unnatural amino acids as described herein.

It is important to note that a key advantage of the genetically-modified AAV of the present invention is that the UAAs incorporated into the minor capsid proteins are at low levels, so their production efficiency is not compromised) significantly affected) by UAA incorporation. Thus, the AAV is packaged/assembled with infectivity titers substantially comparable to wild-type virus.

The variant capsid protein of the genetically-modified AAVs of the present invention can comprise one, or more mutated amino acid residues of VP1, VP2 or both VP1 and VP2 wherein the mutated amino acid residue site incorporates an unnatural amino acid (UAA). Examples of UAAs suitable for use in the present invention are shown in FIG. 1, showing formulas 1-12 (upper line 1 formulas 1-6, left to right and second line formulas 7-12 left to right, including length of carbon chain extensions and substitutions. More specifically, unnatural amino acids suitable for use in the present invention can be selected from the group consisting of phenylalanine analogs; tyrosyl analogs; tryptophanyl analogs; or lysyl analogs. In particular the analogs are selected from the group consisting of: p-benzoylphenylalanine (pBpA); O-methyltyrosine (OMeY); 5-azidotryptophan; 5-propargyloxytryptopha; 5-aminotryptophan, 5-methoxytryptophan; 5-0-allyltryptophan; 5-bromotryptophan; azido-lysine (AzK) or Nε-acetyllysine (AcK)); C5Az, LCA; Nεacetyllysine (AcK); cyclopropene amino acid, N^{ε}-(1-methylcycloprop-2-enecarboxamido)-lysine (CpK); 5-hydroxy-tryptophan (5-HTP);LCA1k; DiaazK and LCKet.

Alternatively, the mutation of the variant minor capsid protein of the genetically-modified AAV can be a natural amino acid residue, such as a cysteine or a selenocysteine. Specifically, the natural amino acid residue is different than the amino acid residue located at the same position of the wild-type AAV.

The genetically-modified AAV of the present invention can further comprise a bioconjugation handle. Such a bioconjugation handle can be covalently attached to the mutated natural or unnatural amino acid residue of the variant minor capsid protein in a site-specific manner. As described herein, 5 to 10 bioconjugation handles can be specifically incorporated into the minor capsid proteins, thus resulting in a genetically-modified AAV with 5-10 bioconjugation handles per fully-assembled AAV capsid. Again, the genetically-modified AAV comprising at least one variant minor capsid protein VP1, VP2 or both VP1 and VP2, retains infectivity of target cells comparable to wild-type AAV under similar conditions, such as cell culture conditions. For example, the mutated amino acid can be conjugated with a chemical or biological entity (e.g., a biological entity can comprise a protein, peptide, nucleic acid, lipid or carbohydrate).

Specifically encompassed by the present invention, is a genetically-modified infectious AAV comprising a capsid comprising variant capsid proteins. For example, the variant capsid protein can comprise SEQ ID NO:1, or a sequence with at least about 80% sequence identity with SEQ ID NO:1, wherein the sequence is mutated to delete VP1, VP2 or both VP1 and VP2. Mutations of the capsid of the genetically-modified AAV can be at position(s), for example, 263, 454, 456, 587 and/or 588 wherein the mutated positions are relative to the wild-type VP1 sequence. The VP1 sequence can also be mutated at positions 263, 454, 456 and 588 but not 587. Alternatively, the VP1 sequence can be mutated at positions 263, 454, 456 but not 585 nor 588.Characteristics of the infectious genetically-modified AAV of the present invention are infectivity substantially comparable to the infectivity of wild-type AAV under comparable conditions, packaging of AAV with titers substantially comparable to wild-type AAV under comparable conditions, or both infectivity and packaging comparable to wild-type AAV.

In one embodiment of the present invention the mutated amino acid residue(s) of the modified AAV is functionalized or conjugated with a chemical or protein entity (also referred to herein as a bioconjugation handle or group). As described herein, the chemical or protein entity is selected from the group consisting of probes, small molecule ligands, peptides, cyclic peptides, nucleotides, polymers, proteins, or a virus conjugate. In a particular embodiment, the genetically-modified infectious AAV comprises a chemical or protein entity, wherein the entity is a cyclic peptide cRGD polyethylene glycol (PEG).

In another embodiment of the present invention, the mutated amino acid residue site of the variants VP1 or VP2 incorporates a naturally-occurring amino acid. In particular, the naturally-occurring amino acid is cysteine or selenocysteine.

The genetically-modified infectious AAV of the present invention comprising the variant capsid protein(s) (for example, the genetically-modified AAV comprising a mutated capsid protein functionalized with a cRGD peptide) can be “re-targeted” from binding to, or recognizing or interacting with, its native cognate cellular receptor to bind to, or recognize or interact with a non-cognate targeted cell. As described herein any number of retargeting groups attached per capsid dramatically affects the properties of the resulting AAV conjugates. This underscores the importance of controlling the site and the stoichiometry for altering the properties of AAV vectors by chemical modification. As described herein, it is now possible for precise, selective chemical modification of AAV capsids with unprecedented control over both the site and the stoichiometry (how many modifications are introduced per capsid). Such control will be extremely important for the development of engineered AAV vectors with more therapeutically desirable properties.

Also encompassed by the present invention is a method of producing/making an infectious genetically-modified AAV, wherein the AAV comprises a variant AAV capsid protein, wherein VP1, VP2 or both VP1 and VP2 comprise one, or more mutated amino acid residue sites, relative to wild-type VP1, VP2 or both VP1 and VP2. In general, the method comprises providing competent host cells in culture, transfecting the cultured cells with one, or more, plasmids comprising 1) an AAV variant VP3 that does not express VP1 or VP2 or neither VP1 and VP2; 2) a variant VP1, VP2 or both VP1 and VP2 with a suitable promoter that do not express VP3; 3) additional factors required for AAV expression ; 4) providing the required unnatural amino acid and 5) providing a plasmid encoding an engineered aminoacyl-tRNA synthetase/tRNA pair that selectively charge the unnatural amino acid in response to a stop codon. The cells and plasmids are cultured under conditions sufficient for expression of the plasmid genes and assembly of the AAV, thereby producing an infectious, genetically-modified AAV comprising a variant capsid protein, wherein VP1, VP2 or both VP1 and VP2 are mutated at one, or more amino acid residues relative to the wild-type AAV.

More specifically, the method comprises the following steps of providing competent host cells in culture; and co-transfecting the cultured cells with plasmids/constructs comprising the required sequences for assembly and expression of the genetically-modified infectious AAV. One mRNA encodes VP1, VP2 or VP3 with different start codon sites (see FIG. 2). If a start site is mutated then expression of VP1, VP2 or VP3 can be controlled by expressing separate mRNAs for each capsid protein, and the capsid proteins can be selectively mutated yet the mutated minor capsid proteins VP1 and/or VP2 and the wild-type can be assembled resulting in an infectious genetically-modified AAV.

For example, as described herein, an unnatural amino acid can be incorporated site-specifically into VP1 and/or VP2 by co-transfecting competent host cells with plasmids encoding an engineered tRNA synthetase and tRNA pair that charge the unnatural amino acid. More specifically, provided is an orthogonal tRNA/aaRS pair comprising an engineered amino-acyl RNA synthetase (aaRS) and its corresponding tRNA to co-translationally incorporate an unnatural amino acid (UAA) in response to the mutated site of the variant VP1, VP2, VP3 or both VP1 and VP2, and providing the required unnatural amino acid to be incorporated at the mutated site.

Also provided (co-cultured) along with plasmids encoding the mutated VP1 and/or VP2 and wild-type VP3 transcripts are plasmid(s) encoding additional factors required for AAV capsid expression and assembly of the complete capsid (i.e., comprising VP1, VP2 and VP3) in cell culture. The cells, plasmids and UAAs are cultured/maintained under conditions sufficient for expression of the plasmid genes and assembly of the AAV, thereby producing an infectious genetically-modified AAV comprising a variant AAV capsid protein, wherein VP1, VP2 or both VP1 and VP2 are mutated at one, or more amino acid residue sites relative to the wild-type AVV.

The engineered aminoacyl-tRNA-synthetase-tRNA pair can be derived from the E.coli leucyl pair, E. coli tryptophanyl pair, E. coli tyrosyl pair or archea-derived pyrrolysyl pair.

The infectious, genetically-modified AAV can be recovered/harvested from the cell culture and evaluated for biological activity using techniques as described herein and as known to those of skill in the art.

The mutated amino acid residue site of the infectious genetically-modified AAV can incorporate an unnatural amino acid (UAA) analog as shown in FIG. 1, formulas 1-12. More specifically, the unnatural amino acid is selected from the group consisting of phenylalanine analogs; tyrosyl analogs; tryptophanyl analogs; or lysyl analogs. The analogs are selected from the group consisting of: p-benzoylphenylalanine (pBpA); O-methyltyrosine (OMeY); 5-azidotryptophan; 5-propargyloxytryptopha; 5-aminotryptophan; 5-methoxytryptophan; 5-O-allyltiyptophan; 5-bromotryptophan; azido-lysine (AzK) or Nε-acetyllysine (AcK); C5Az; LCA; Nε-acetyllysine (AcK); cyclopropene amino acid, N^{ε}-(1-methylcycloprop-2-enecarboxamido)-lysine (CpK); 5-hydroxy-tryptophan (5-HTP),LCAlk, DiaazK and LCKet.

Alternatively, the mutated amino acid residue site of the variant capsid protein incorporates a naturally-occurring amino acid, and the naturally-occurring amino acid is cysteine or selenocysteine.

The mutated amino acid of the variant capsid protein can be functionalized with a chemical or protein entity, wherein the entity is selected from the group consisting of probes, small molecule ligands, peptides, cyclic peptides, nucleotides, polymers, proteins, or a virus conjugate. In one embodiment the entity functionalizing the mutated amino acid of the variant capsid protein of the infectious genetically-modified AAV is a cyclic peptide cRGD.

The infectious genetically-modified AAV can comprise the mutated VP1 amino acid sequence comprises SEQ ID NO:1, or a sequence with at least about 80% sequence identity with SEQ ID NO:1, wherein the variant VP1 capsid protein is mutated at one, or more position(s) located at 263, 454, 456, 587 and/or 588 of the VP1 sequence and relative to the wild-type VP1 sequence. The VP1 sequence can also be mutated at positions 263, 454, 456 and 588 but not 587. Alternatively, the VP1 sequence can be mutated at positions 263, 454, 456 but not 585 nor 588.

The infectious genetically-modified AAV can comprise the mutated VP2 amino acid sequence comprises SEQ ID NO:2, or a sequence with at least about 80%, 85%, 90%, 95 or 99% sequence identity with SEQ ID NO:2.

The infectious genetically-modified AAV can comprise the VP3 amino acid sequence comprises SEQ ID NO:3, or a sequence with at least about 80%, 84%, 90%, 95% or 99% sequence identity with SEQ ID NO:3.

Also encompassed by the present invention are therapeutic or antigenic compositions comprising a genetically-modified AAV as described herein and further comprising one, or more therapeutic or antigenic gene constructs suitable for gene therapy or vaccination (e.g., eliciting an immune response in a subject). If the composition is a vaccine composition comprising an antigen, the composition can further comprise an adjuvant.

Methods of treating a disease or condition in a subject are also encompassed herein. For example, the method of treatment can comprise administering a therapeutic composition to the subject, wherein the composition comprises an AAV vector as described herein and a gene construct encoding a protein or peptide in a therapeutic amount capable of decreasing or alleviating the disease or condition in the subject. Use of a genetically-modified AAV vector of the present invention in therapeutic compositions can be particularly useful for treating cancer, or a disease such as cystic fibrosis of sickle-cell anemia, where providing a replacement gene encoding a functional protein in a targeted manner would decrease, or completely alleviate, the cancer or disease symptoms.

Another example is a method of eliciting an immune response in a subject, the method comprising administering an antigenic/vaccine composition to the subject, wherein the antigenic composition comprises the genetically-modified AAV vector of the present invention and a gene construct encoding an antigenic protein or peptide capable of eliciting an immune response in the subject. Such a method of using the AAV vector of the present invention would specifically target immune cells capable of mounting an immune response.

Kits comprising the genetically-modified AAV of the present invention are also encompassed herein. Such kits can include a suitable vial containing the genetically-modified AAV as described herein, for example a vial of the AAV in a sterile diluent, as well as vials/containers of supplemental components, for example for propagating the AAV in cell culture. An instructional pamphlet can also be included in the kit.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. The patent or application file contains at least one drawing executed in color. Copies of this patent or patent application publication with color drawings(s) will be provided by the Office upon request and payment of the necessary fee. Of the drawings:

FIG. 1 shows the examples of the structures of the natural and the unnatural amino acids that can be incorporated into the capsid of AAV in a controlled stoichiometry. Specifically shown are UAA analogs comprising the formulas 1-12 (upper line 1 formulas 1-6, left to right and second line formulas 7-12 left to right, including length of carbon chain extensions and substitutions.

FIG. 2 shows the scheme for introducing mutations (natural or unnatural amino acids) selectively into VP1, or VP2, or VP1 and VP2. Three capsid proteins are expressed from the same open reading frame Cap via the use of alternative splicing and start codon usage. Mutations have been engineered at the translation origin (demonstrated by red crosses) that prevent the expression of VP1, VP2, or VP3 from this ORF. The missing minor capsid protein(s) can then be supplied in trans, driven by a strong promoter such as CMV. Separating the expression of VP1, VP2, or VP1+VP2 from the rest of the capsid proteins makes it possible to selectively mutate these without affecting the other capsid proteins.

FIG. 3 shows packaging of AAV2 in HEK293T cells using constructs described in FIG. 2. AAV with variant capsid proteins have comparable yields relative to the original system.

FIG. 4 shows the infectivity of the packaged, tittered viruses produced in FIG. 3 at constant titer measured by their ability to deliver and express an EGFP reporter gene in HEK293T cells.

FIG. 5 shows selective unnatural amino acid mutagenesis of individual minor capsid proteins in AAV capsid and their use to chemically attach a fluorophore.

FIG. 6 shows the number of retargeting ligands attached to the AAV capsid dramatically affects its retargeting efficiency.

FIGS. 7A-7C show precise labeling of AAV at engineered cysteine residues.

FIGS. 8A-C show the amino acid sequences for AAV isoform VP1( SEQ ID NO:1). FIGS. 8B and 8C disclose SEQ ID NO: 2 and SEQ ID NO: 3, respectively.

FIG. 9 shows the amino acid sequence of AAV capsid protein isoform VP2. (SEQ ID NO:2).

FIG. 10 shows the amino acid sequence of the AAV capsid protein isoform VP3. (SEQ ID NO:3).

FIGS. 11A-C shows the selective incorporation of the UAA C5Az either VP1 or VP2 or VP1+VP2Fig.

FIGS. 12A-C shows the results of LCA incorporated into VP1.

FIGS. 13A-C shows the results of incorporation of CpK into VP1.

FIGS. 14A-C shows the results of incorporation of 5HTP into VP1.

FIGS. 15A-B demonstrates incorporation of several other unnatural amino acids, LCAlk, DiazK and LCKet, into VP1.

FIGS. 16A-B shows the results of selective PEGylation of AAV at VPI site 454.

FIG. 17 shows the nucleic acid sequence encoding RC2-VP1-del. (SEQ ID NO:4) The location of the mutation is shown in red.

FIG. 18 shows the nucleic acid sequence encoding RC2-VP2-del. (SEQ ID NO:5) The location of the mutation is shown in red.

FIG. 19 shows the nucleic acid sequence encoding RC2-VP12-del. (SEQ ID NOL6) The location of the mutation is shown in red.

FIG. 20 shows the nucleic acid sequence encoding CMV-VP1-delVP23. (SEQ ID NO:7) The locations of the mutations are shown in red.

FIG. 21 shows the nucleic acid sequence encoding CMV-VP2-delVP3. (SEQ ID NO:8) The locations of the mutations are shown in red.

FIG. 22 shows the nucleic acid sequence of CMV-VP1-VP2-delVP3. (SEQ ID NO:9) The locations of the mutations are shown in red.

FIG. 23A is the plasmid map of pIDTsmart-ITR-GFP-4xEcLtR-LeuRS. FIG. 23B shows the nucleic acid sequence of the plasmid. (SEQ ID NO:10).

FIG. 24A shows the plasmid map of pIDTsmart-TrpRS-8xWtR-ITR-GFP. FIG. 24B shows the nucleic acid sequence of the plasmid. (SEQ ID NO:11).

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should be noted that this invention description is not limited to these specific methodologies, compositions, cell lines/biological systems, or any other standard protocol. The present invention discloses a general platform for producing AAV vectors that can be chemically functionalized using chemo-selective reactions with control over site and stoichiometry of modification, the cell type used for virus packaging, identity of the engineered aminoacyl-tRNA synthetase (aaRS) or tRNA, or UAAs or natural amino acids, the chemical reaction used for introducing the modification, etc., can vary as the technology in this field and in this work advance.

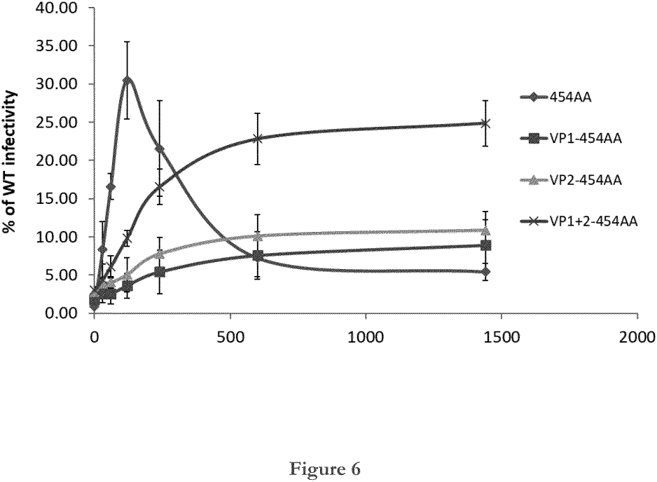

Previously, incorporation of UAAs into the AAV capsid has been directed to all 60 capsid proteins. Since the three overlapping capsid proteins are expressed from the same open reading frame (ORF) Cap via alternative splicing and start codon usage, it has not been possible to selectively modify a subset of these proteins. However, subsequent chemical modification of all of the 60 capsid proteins perturbs the infectivity of the virus (e.g., FIG. 6, blue trace). While the mechanism of this perturbation is poorly understood, it is reasonable to believe that the molecular processes associated with the complex entry pathway of the virus can be affected by over-modification of the capsid. Additionally, it is desirable to be able to create AAV vectors into which a defined number of chemical modifications can be introduced in a site-specific manner. Indeed, retargeting experiments demonstrate that there is an optimal number of ligands per capsid needed for efficient retargeting (FIG. 6). It is possible to control the degree of modification of an AAV capsid with 60 UAA-handles by controlling the concentration of the modifying reagent or reaction time (FIG. 6). However, this leads to a heterogeneous mixture of capsids in different states of modification. The ability to create homogeneous AAV conjugates where a defined number of modifications are introduced per capsid is critical for gene therapy.

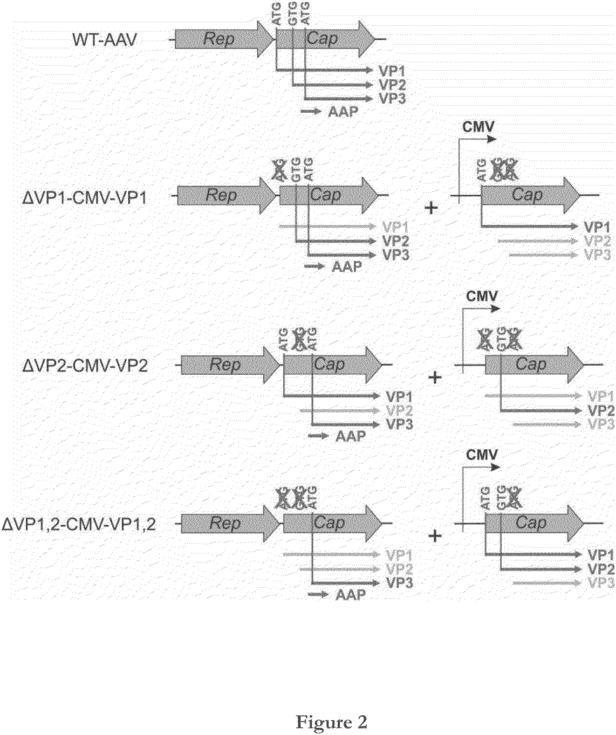

In the AAV capsid, the three capsid proteins VP1, VP2, and VP3 are incorporated at a roughly 1:1:10 stoichiometry. Consequently, approximately 5 copies of each of the two minor capsid proteins, VP1 and VP2, are present in the capsid, with 10 copies of VP3, for a total of 60 copies of capsid proteins per virus particle. The ability to selectively introduce engineered, modifiable natural or unnatural amino acid residues into these minor capsid proteins provides an avenue to introduce a controlled number of handles per capsid. However, since the AAV capsid genes are all encoded in the same ORF and expressed by alternative splicing and start-codon usage, it is challenging to mutate one capsid protein without affecting the others. The present invention describes a method to separately express the minor capsid proteins (VP1, or VP2, or VP1+VP2) by introducing mutations at the translation origins of VP1 and/or VP2, such that these are not expressed from the native Cap ORF. Next, the missing capsid protein can be expressed in trans from a strong promoter, for example, the CMV promoter as described herein. Expression of the undesired capsid proteins (e.g., VP3) from this second ORF is also similarly eliminated by mutating translation origins (FIG. 2). This platform provides the ability to express any combinations of the three capsid proteins from one ORF and separating the expression of a chosen third from a second ORF, making it possible to selectively engineer any subset of the three capsid proteins.

Using the platform described herein, it is now possible to selectively introduce unique non-coding codons (such as nonsense, 4-base, or unnatural base-pair-containing codons) into any combination of the three capsid proteins by site-directed mutagenesis. When such a mutant Cap is co-expressed with a suitable engineered aminoacyl-tRNA synthetase (aaRS)/tRNA pair that can decode the unique codon, an UAA residue can be incorporated into these sites.

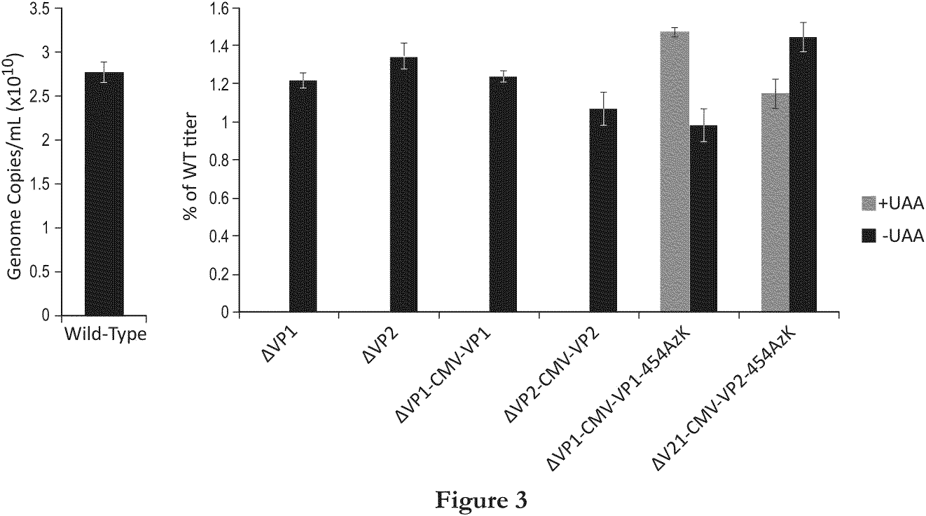

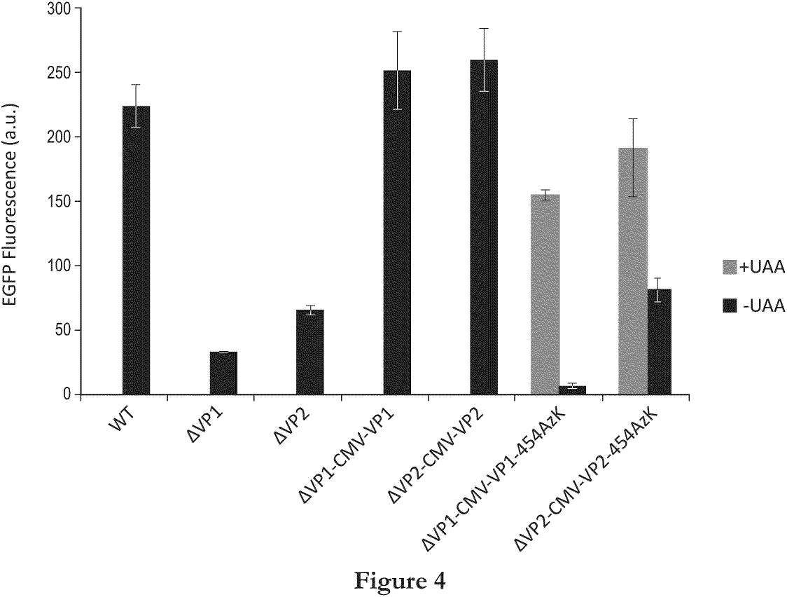

In one embodiment of this invention, the UAA AzK was incorporated at residue T454 (VP1 numbering) of VP1 or VP2 or VP1+VP2 (FIG. 3). Using a pyrrolysyl-tRNA synthetase/tRNA pair. The resulting viruses were packaged at an efficiency comparable to the wild-type virus and had comparable infectivity, demonstrating these modifications are well-tolerated (FIG. 3 and FIG. 4). The importance of controlling the number of modifications per capsid was demonstrated by the differential behavior of AAV where the UAA was incorporated at 5 copies per capsid (mutation of either VP1 or VP2 alone), or 10 copies per capsid (mutation of both VP1+VP2), or 60 copies per capsid (all three capsid proteins were mutated), and a retargeting ligand was subsequently attached to these UAA handles (FIG. 6). AAV with 60 attachment handles enabled efficient retargeting at intermediate modification states, but upon full modification, all infectivity was lost. 5 or 10 modifications per capsid were well-tolerated, but 10 retargeting ligands per capsid was needed for efficient retargeting (FIG. 6).

It should be noted that any other combination of capsid proteins, and any site within these proteins, can be engineered using this strategy. Especially well-suited for mutation is the N-terminus of the capsid proteins as this section of the capsid protein is essentially internal when folded into the functional capsid, whereas the C-terminus of the capsid protein is exposed.

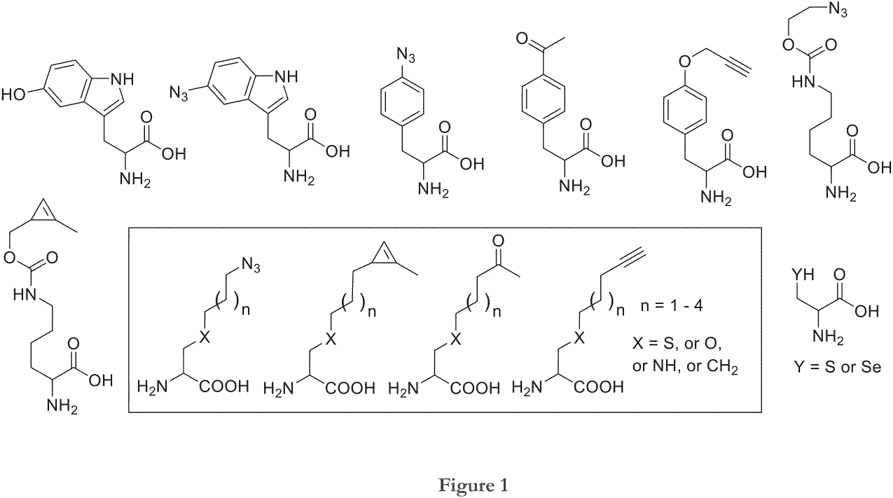

Additionally, other aaRS/tRNA pairs can be used to (including, but not limited to, bacterial tyrosyl, tryptophanyl, and leucyl-tRNA synthetase/tRNA pairs) and incorporate any other unnatural amino acids (illustrative examples shown in FIG. 1 and described herein). This technology can also be extended to any other natural serotype of AAV as well as engineered and evolved variants of AAV.

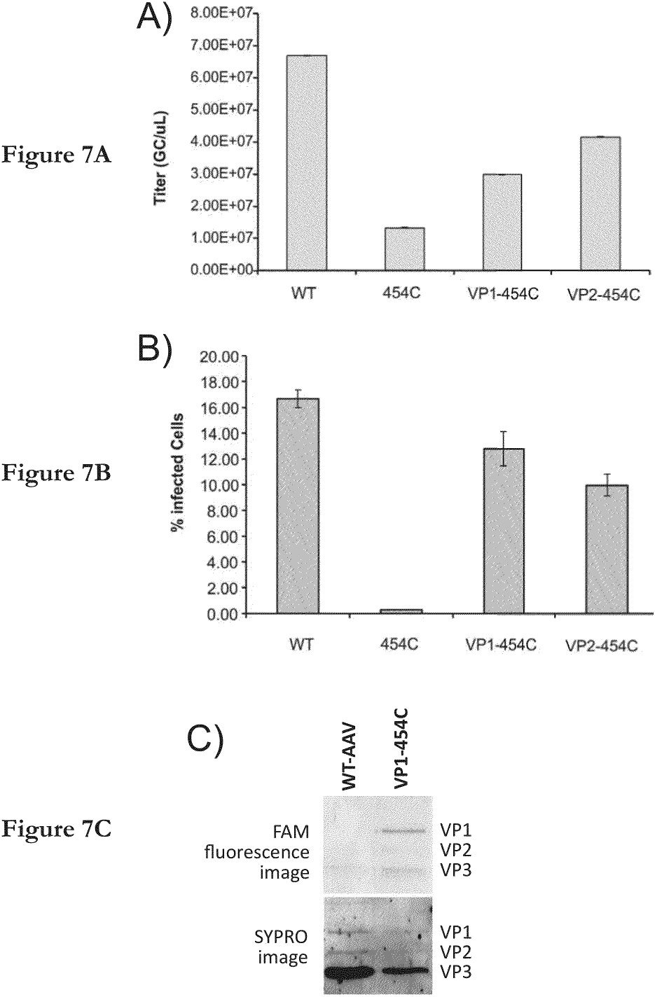

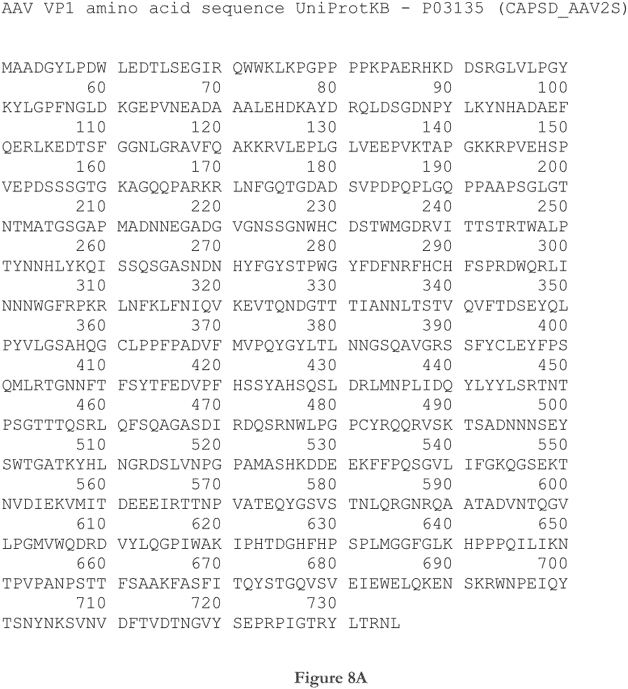

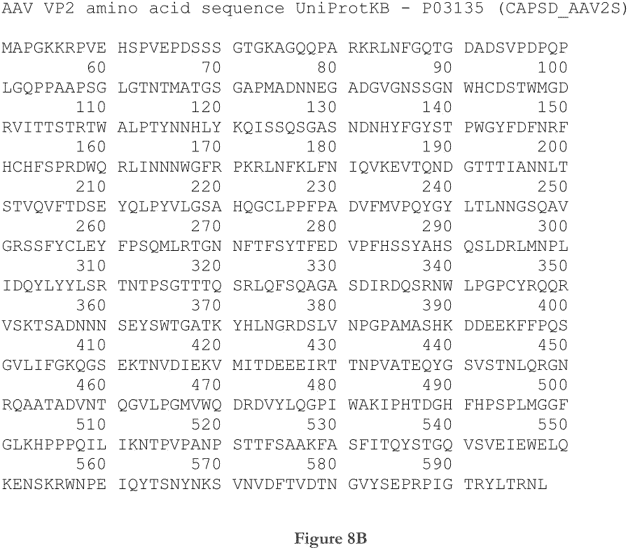

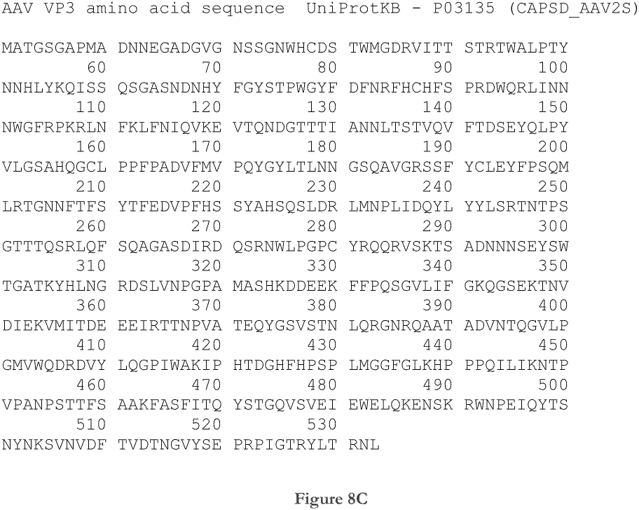

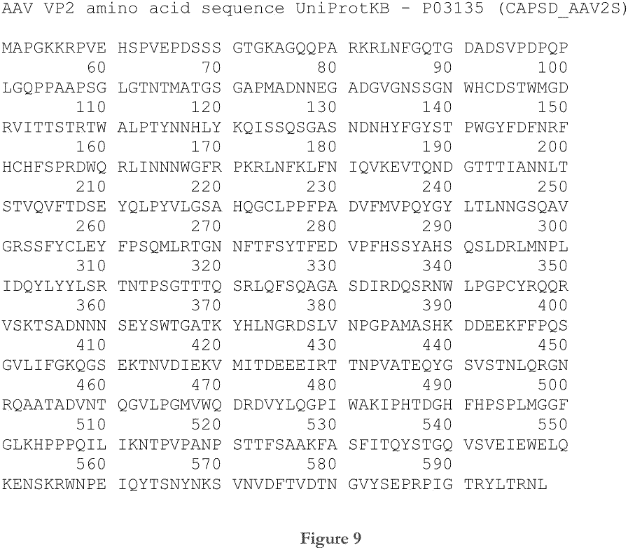

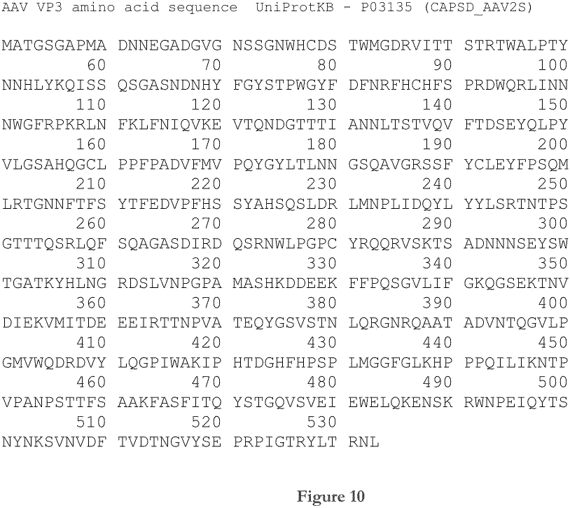

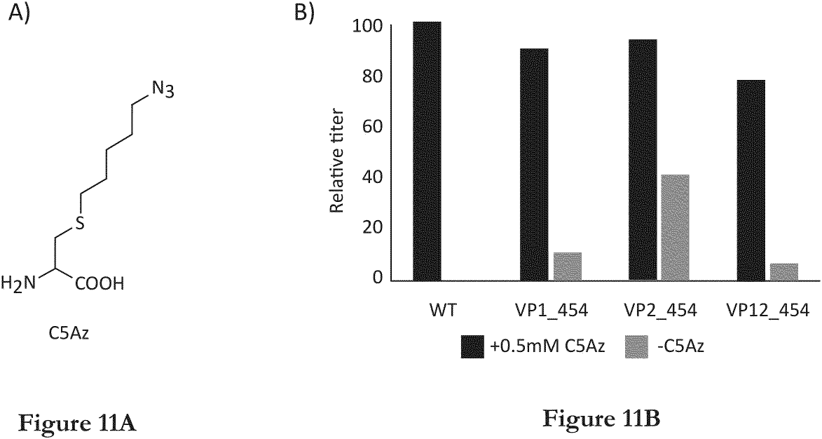

Cysteine and selenocysteine are natural amino acid residues found in proteins. Because of their low abundance and unique reactivity, these can be used for site-selective bioconjugation reactions. In another embodiment of this invention, engineered surface-exposed cysteine residues can be introduced into the minor capsid proteins (FIGS. 7A and 7B). Even though the same mutation is not well-tolerated when introduced to all 60 capsid proteins, leading to low titer and poor infectivity, robust virus packaging and infectivity was observed when surface exposed cysteine residue was introduced only to VP1 or VP2 at the T454 site. The ability to selectively modify the engineered cysteine residue on the minor capsid protein was also demonstrated (FIG. 7C).

Because of the complexities associated with the assembly and the cell-entry process, for which the AAV capsid has been optimized through evolution, it frequently resists attempts at engineering the capsid protein through natural/unnatural amino acid mutagenesis, loop insertion, protein fusion etc. However, engineering just the minor capsid proteins is tolerated significantly better as it introduces much less overall perturbation to the capsid overall. This is illustrated by the tolerance of the engineered cysteine residue at the minor capsid protein, but not everywhere (FIG. 7). Thus, this invention provides an opportunity to introduce more aggressive engineering to alter the properties of the AAV capsid through engineering the minor capsid protein. In addition to natural and unnatural amino acid mutagenesis, this invention can also be used to introduce peptide and protein fusions and insertions into the minor capsid proteins that either directly provide a beneficial trait (e.g., binding a certain target), or can be selectively modified through chemical or enzymatic reactions (e.g., biotinylation tag, SNAP or HALO tag, etc.).

This invention allows the introduction of a defined number of engineered residues (natural or unnatural) per capsid by selectively mutagenizing VP1, or VP2, or VP1 plus VP2. Further control over the number of engineered sites can be achieved by introducing more than one engineered residue into VP1, or VP2, or VP1+VP2. As described herein, various sites of the VP1 capsid protein have been selectively mutated such as 263, 454, 456, 587 and 588 (the numbering corresponds to the amino acid residues of wild-type VPI). The platform can be extended to any packaging platform including, but not limited to, mammalian cells, insect cells, and cell-free translation/packaging systems.

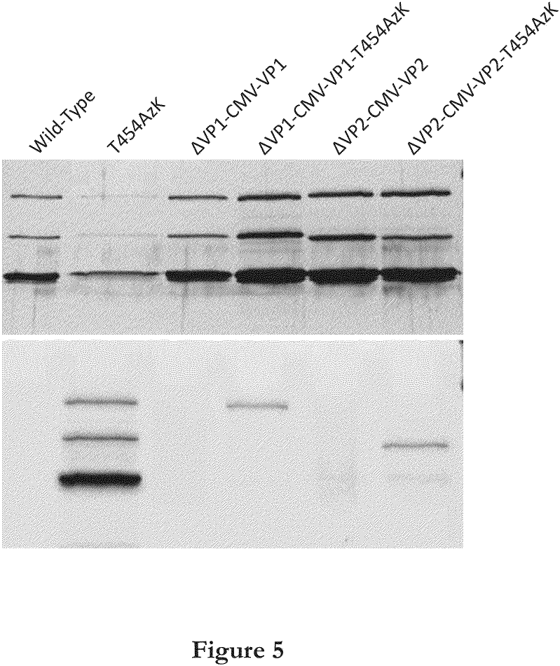

The invention enables the incorporation of numerous natural and unnatural amino acid residues into AAV with control over site and copy number, with a wide variety of different chemistries which can be used to chemo-selectively attach various entities. In one embodiment of this invention, an azido-containing UAA was introduced selectively into the minor capsid proteins of AAV, followed by their conjugation to a fluorophore or a retargeting ligand using strain-promoted azide-alkyne click reaction (FIG. 5 and FIG. 6). In another embodiment, an engineered cysteine residue in a minor capsid protein was introduced and subsequently conjugated to a fluorophore using cysteine-maleimide coupling reaction. Any other chemo-selective conjugation reaction can be applied for the capsid modification including, but not limited to, inverse-electron demand Diels-Alder reaction between a strained alkene and a tetrazine, or a furan and a maleimide, condensation reaction between an aldehyde/ketone and an amino-oxy/hydrazine groups, chemo-selective rapid azo-coupling reaction (CRACR), oxidative and photocatalyzed coupling reactions, nucleophilic substitution/addition by cysteine or selenocysteine residue to various electrophiles, etc.

The methods described herein can be used to attach a wide variety of entities including, but not limited to, probes (fluorescent, radioactive, MRI, luminescent, etc.), small molecule ligands, peptides, cyclic peptides, nucleotides (DNA, RNA, LNA, PNA, etc.), polymers (such as PEG), carbohydrates (e.g., sialic acids, etc.), proteins (e.g., enzymes, nanobodies, antibodies, etc.), another AAV of the same or different serotype, etc. Such attachment can provide AAV conjugates that efficiently retargets to a user-defined receptor, thus altering its tissue tropism. Immune-evading AAV can also be created by site specifically attaching groups (such as PEG, peptides, carbohydrates, or other polymers) that passively protect the capsid from the immune system, or ligands that actively bind inhibitory receptors on immune cells to turn off immune response (such as SIGLEC ligands). It can also be used to attach enzymes on AAV capsids to produce capsids with superior infectivity profile, or conjugate two AAVs with same or different serotypes to create novel class of vectors with expanded cargo capacity as well as novel tropism.

The controlled AAV modification technology descried herein can be used for many applications such as targeting AAV vectors to desired types of cells by attaching retargeting ligands that include, but are not limited to, small molecules, peptides, cyclic peptides, nanobodies, antibodies and antibody fragments; DNA/RNA/PNA aptamers, etc.; optimizing the properties of such conjugates by systematically controlling the attachment site, the number of retargeting groups per capsid, and the chemical properties of the linker; Attenuating the immune response generated by AAV vectors by the controlled attachment of immuno-modulatory entities including, but not limited to, polyethylene glycol and other polymers, carbohydrates (such as sialic acid, etc.), ligands that bind inhibitory receptors on immune cells (such as SIGLEC receptor), etc.

In particular the genetically-modified AAV of the present invention can be used as an enhance vector for gene therapy, Using techniques described herein, the AAV can be genetically-modified to specifically target/direct delivery of a therapeutic or antigenic gene construct to the target cell with enhanced/increased efficacy over non-modified AAV vectors. The cell can be cultured in vitro or in vivo or ex vivo delivery to a subject in need thereof. The term “subject” as used herein can include any animal subject, and in particular includes a mammalian subject such as a human. The human subject can be treated for medical purposes using the AAV gene vector described herein, such as for the treatment of an existing disease, disorder, condition or the prophylactic treatment for preventing the onset of a disease, disorder, or condition or an animal subject for medical, veterinary purposes, or developmental purposes. The methods and compositions of the present invention may be used to treat any type of cancerous tumor or cancer cells. Additionally, the genetically-modified AAV of the present invention can be used in a vaccine composition wherein a nucleic acid sequence encoding an antigenic agent such as a protein or peptide is delivered to the targeted cell along with additional components such as adjuvants wherein an immune response is elicited in the subject.

The gene construct delivered by the genetically- modified AAV is therapeutically effective. A “therapeutically effective” amount as used herein refers to an amount sufficient to have the desired biological effect to produce the desired effect on the underlying disease state (for example, an amount sufficient to inhibit tumor growth in a subject, produce an immune response to an antigen or to inhibit autoimmune disease) in at least a sub-population of cells in a subject at a reasonable benefit/risk ratio applicable to any medical treatment. Determination of therapeutically effective amounts of the constructs/agents used in this invention, can be readily made by one skilled in the art, by the use of known techniques and by observing results obtained under analogous circumstances.

The technology can also be used for conjugating two distinct AAV vectors, of the same or different serotypes, using a bifunctional linker to increase the overall size of the genetic cargo delivered per cell. Such novel conjugates between two different AAV vectors also will have unique tropism, and for conjugating external payloads (such as proteins, small molecules, nucleic acids, probes, etc.) onto the virus capsid to be delivered into cells in vitro and in vivo for research or therapeutic purposes that work independently, or in conjunction with the genetic cargo inside the AAV capsid.

Finally, this technology can be used for the investigation of the entry pathway of AAV capsids into mammalian cells by incorporating groups such as fluorescent probes, photo-crosslinkers, and affinity handles (such as biotin).

Without further elaboration, it is believed that one skilled in the art can, based on the above description, utilize the present invention to its fullest extent. The following specific embodiments and examples are, therefore, to be construed as merely illustrative, and not limitative of the remainder of the disclosure in any way whatsoever.

Incorporation of UAAs into the capsid of AAV requires efficient expression of its capsid protein(s), including the desired UAA modification in a competent cell (e.g., mammalian cell) serving as the host for virus amplification. As described earlier, the site of UAA incorporation can be specified by a stop codon (such as TAG), and the UAA of interest can be delivered by an engineered tRNA/aminoacyl-tRNA synthetase pair, with a cognate anticodon. Consequently, the production of UAA-modified viruses must involve the simultaneous expression of genetic components necessary for virus amplification, as well as those necessary for the amplification of the virus.

To incorporate an UAA into the capsid proteins of AAV, the replacement of several surface exposed endogenous amino acids residues was targeted on the AAV capsid (guided by a crystal structure of the virus particle, see, e.g., Xie, et al., The atomic structure of adeno-associated virus (AAV-2), a vector for human gene therapy” PNAS Aug. 6, 2002: vol. 99: no. 16, p. 10407). Since such capsid mutations can potentially perturb viral infectivity, regions that are known to tolerate alterations were targeted, such as those on the threefold proximal spike. The replacement of essential arginine residues was also targeted in the heparan sulfate receptor binding region, which disrupts native tropism and will facilitate re-targeting. This allows “erasing” the native host cell preference of AAV and rewriting it with targeting agents with precise labeling methodology. For example, positions targeted can reside in the domain conserved among all of the three AAV capsid proteins, leading to their replacement with UAAin all of them. AAV can be produced by transfecting e.g., HEK293T cells with plasmids containing the required elements in the presence of the UAA azido-lysine (“AzK”). In a specific example a Methanosarcina barkeri-derive pyrrolysyl-tRNA synthetase and an M. mazeii derived pyrrolysyl tRNA (TAG suppressor) was used to incorporate the AzK amino acid in response to the stop codon, TAG at T454.

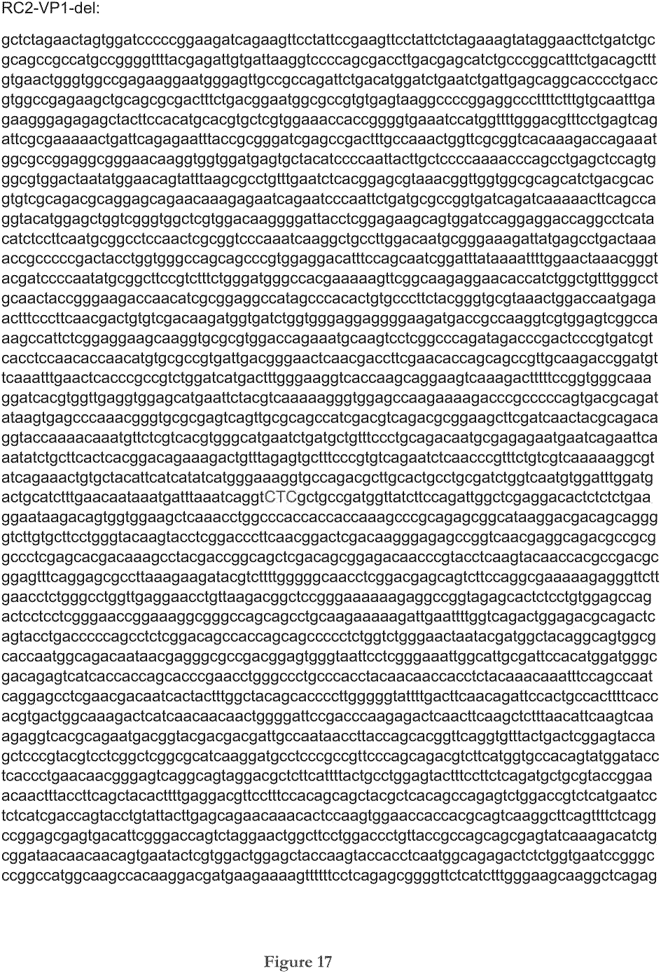

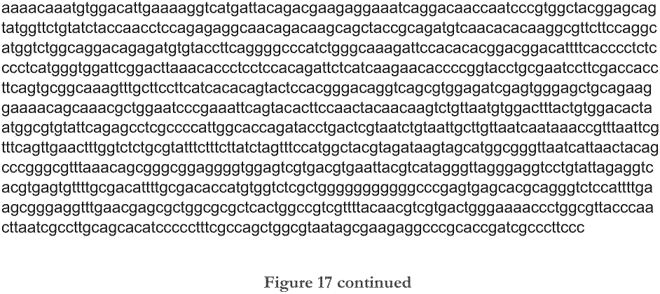

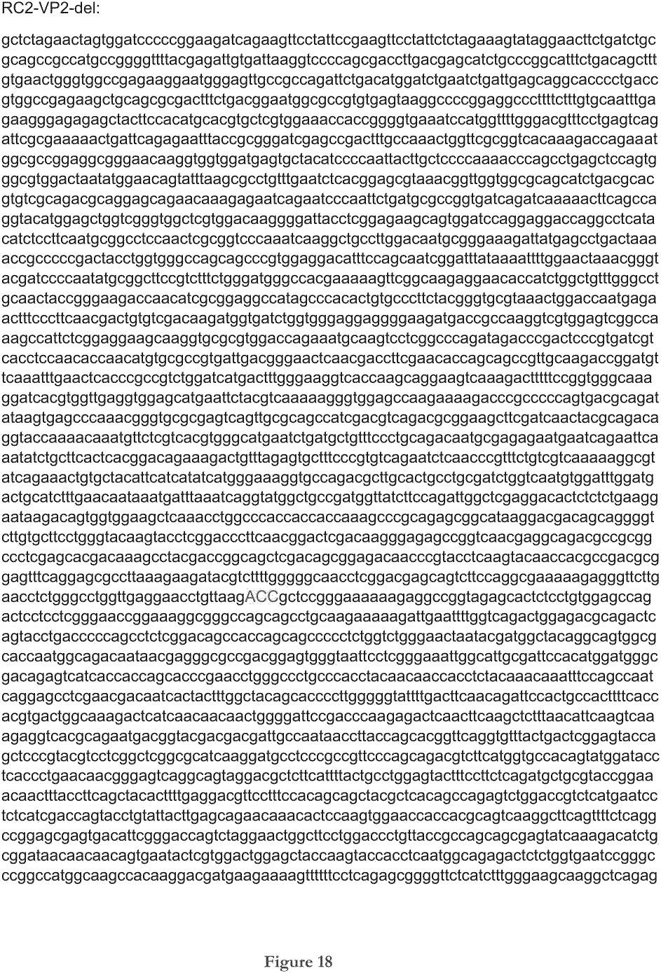

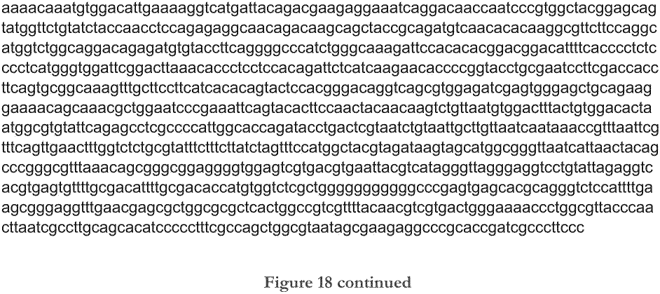

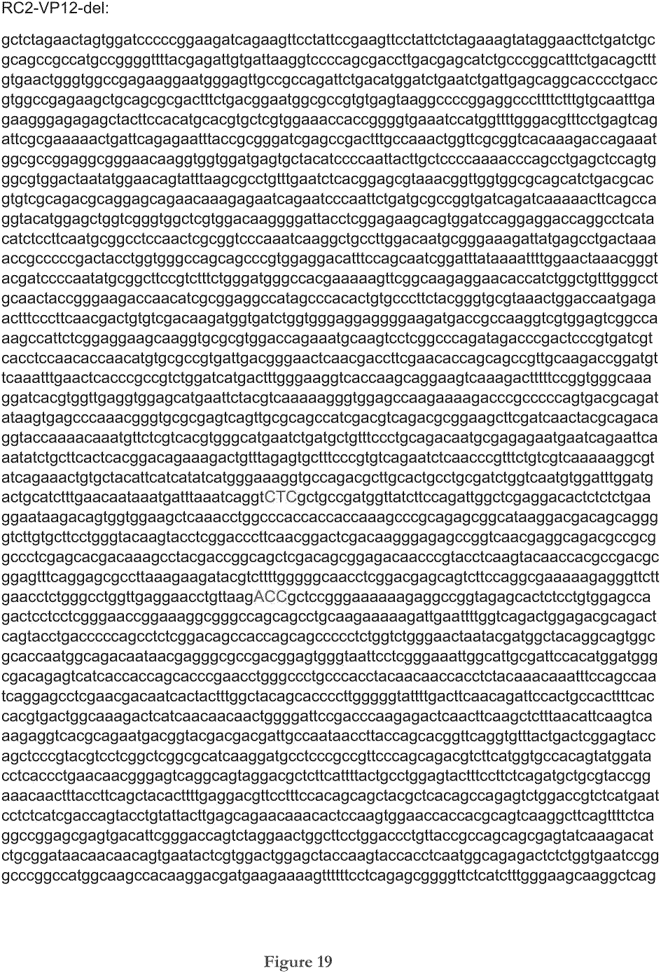

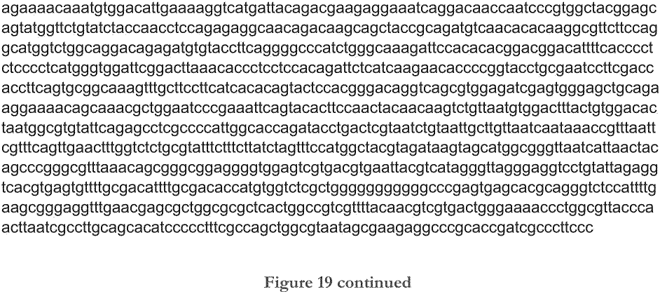

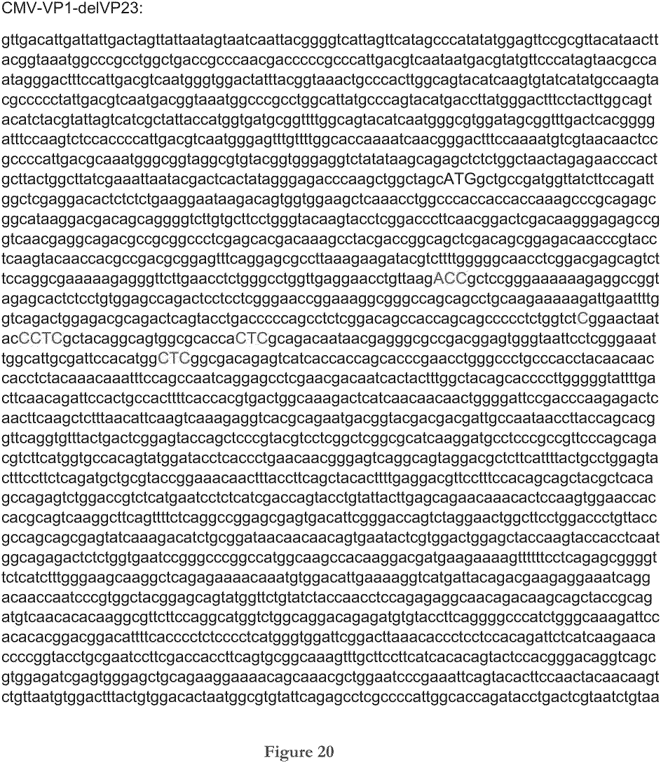

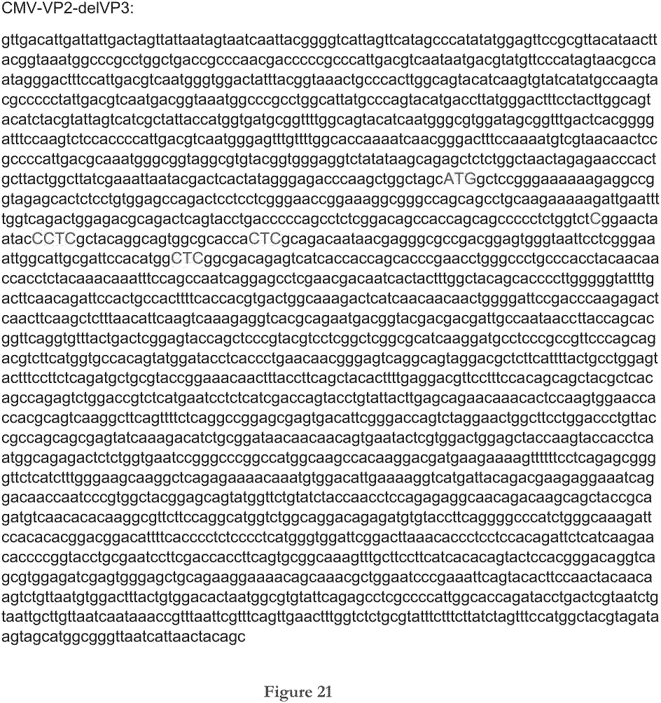

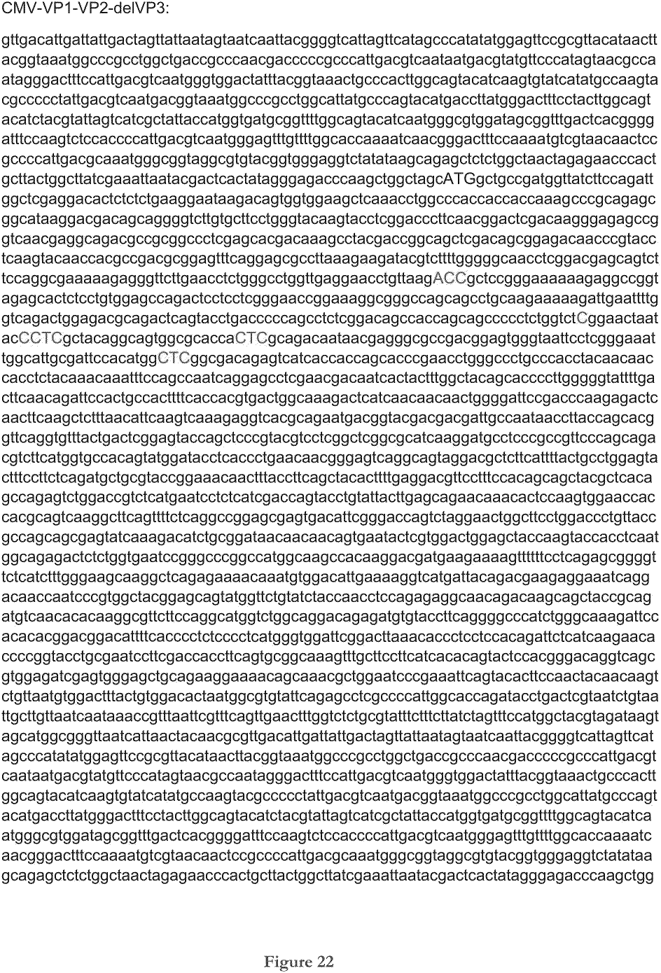

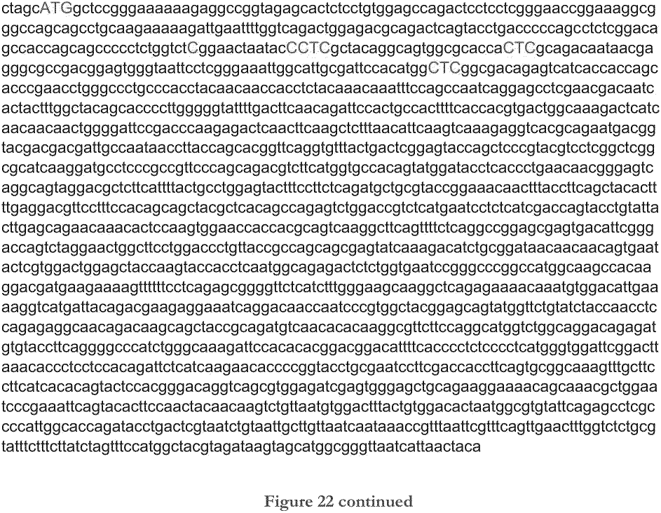

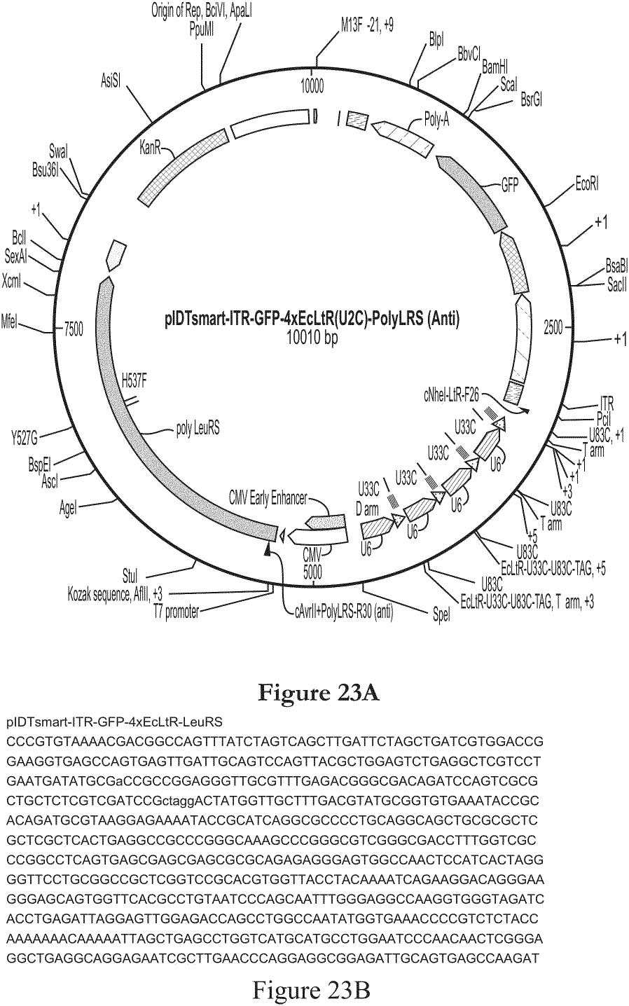

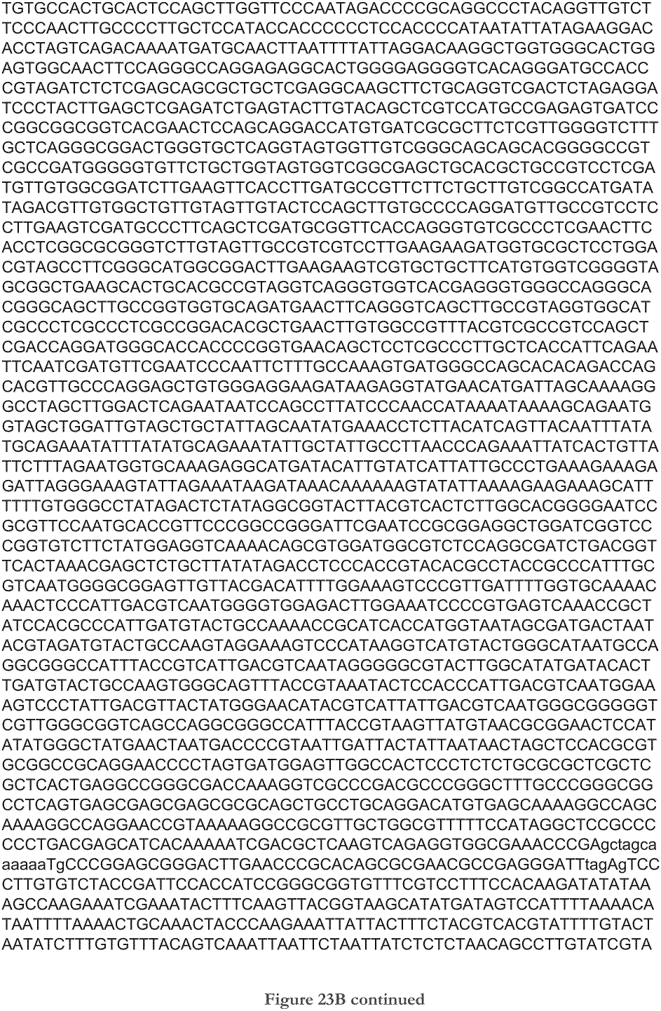

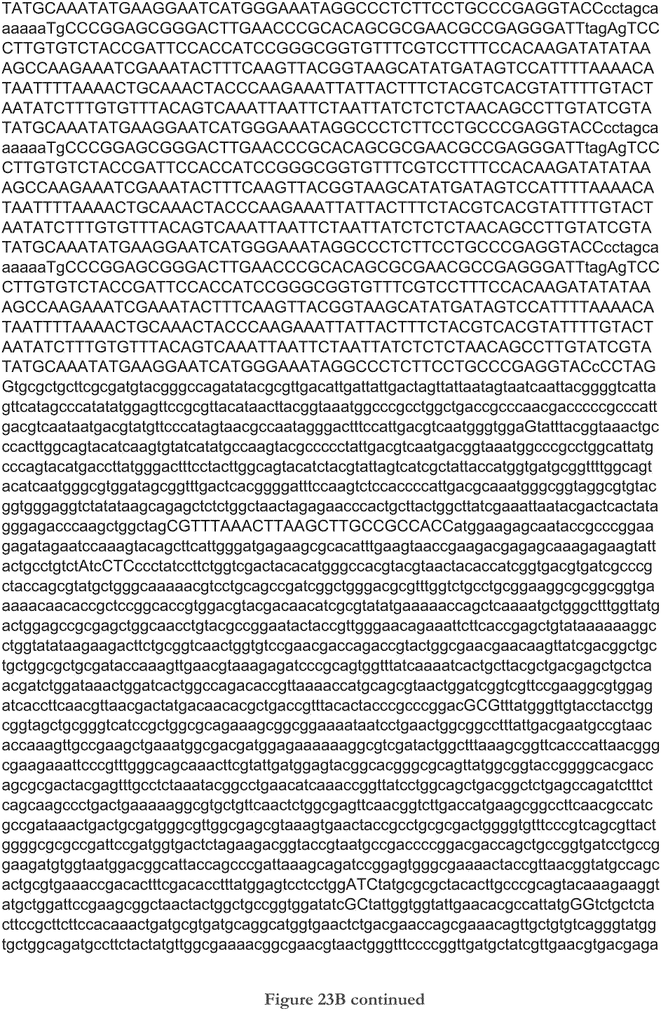

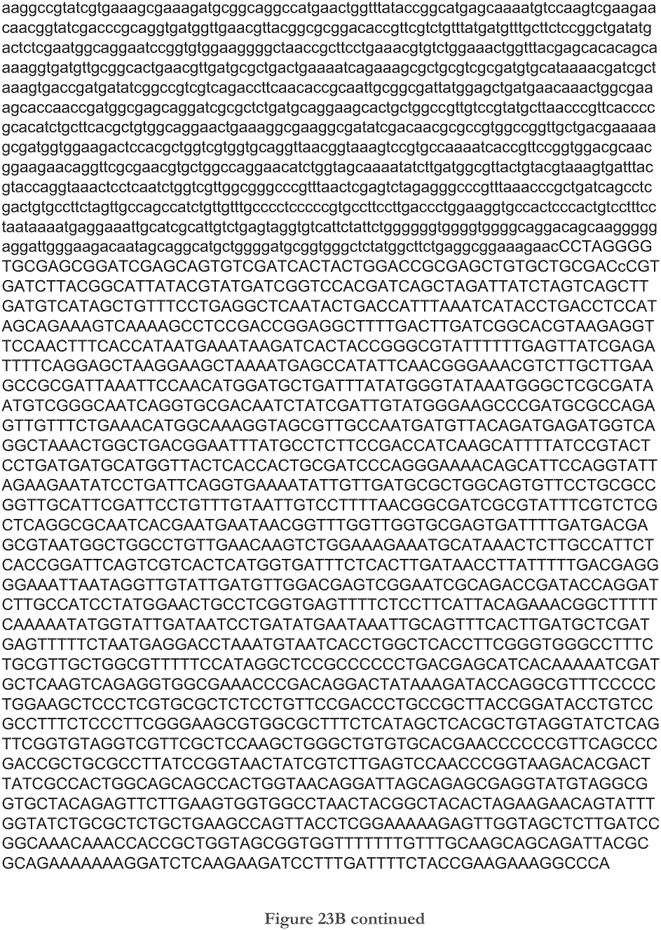

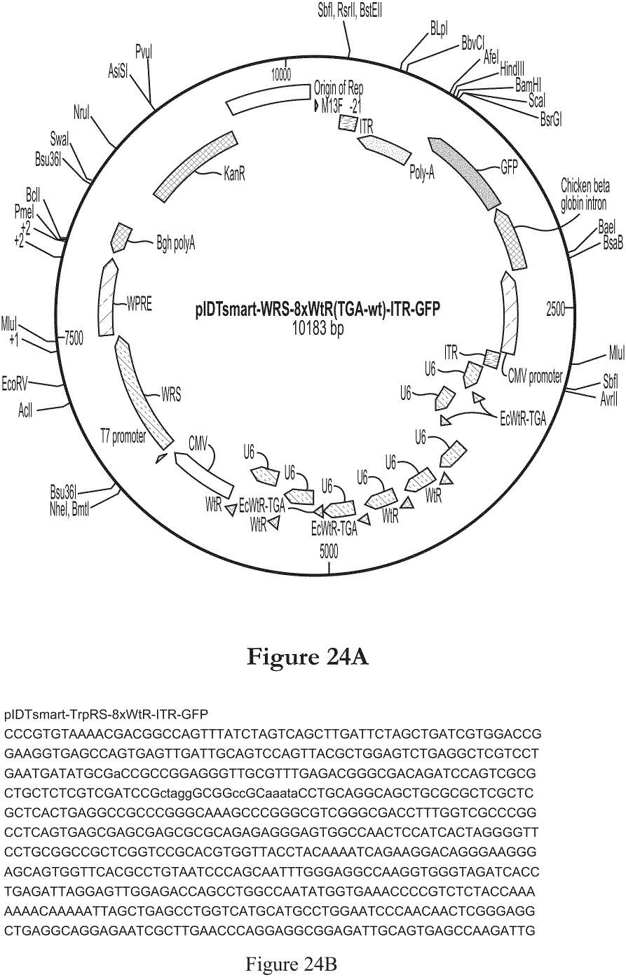

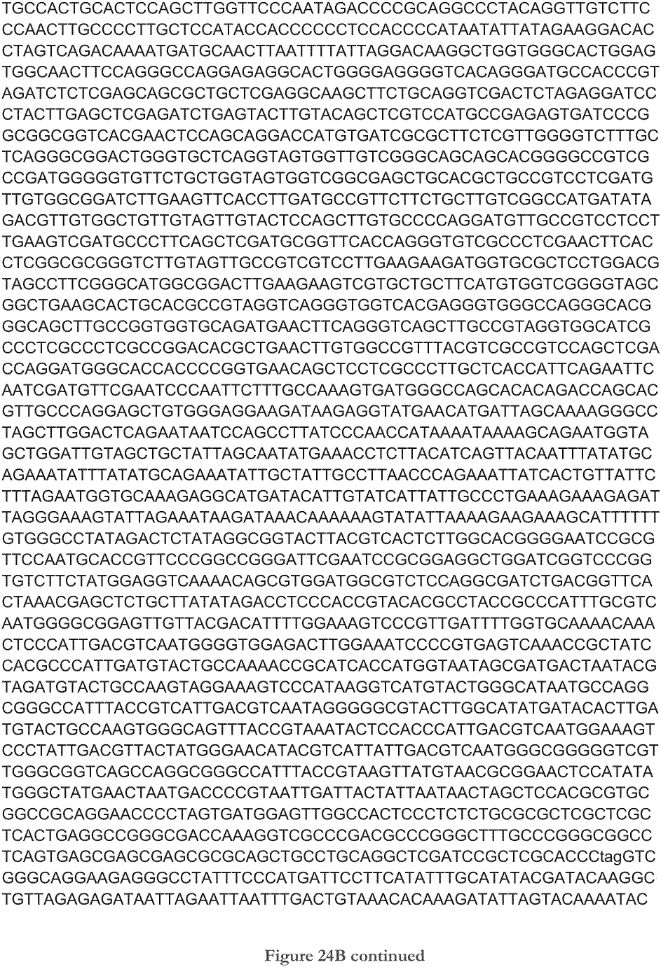

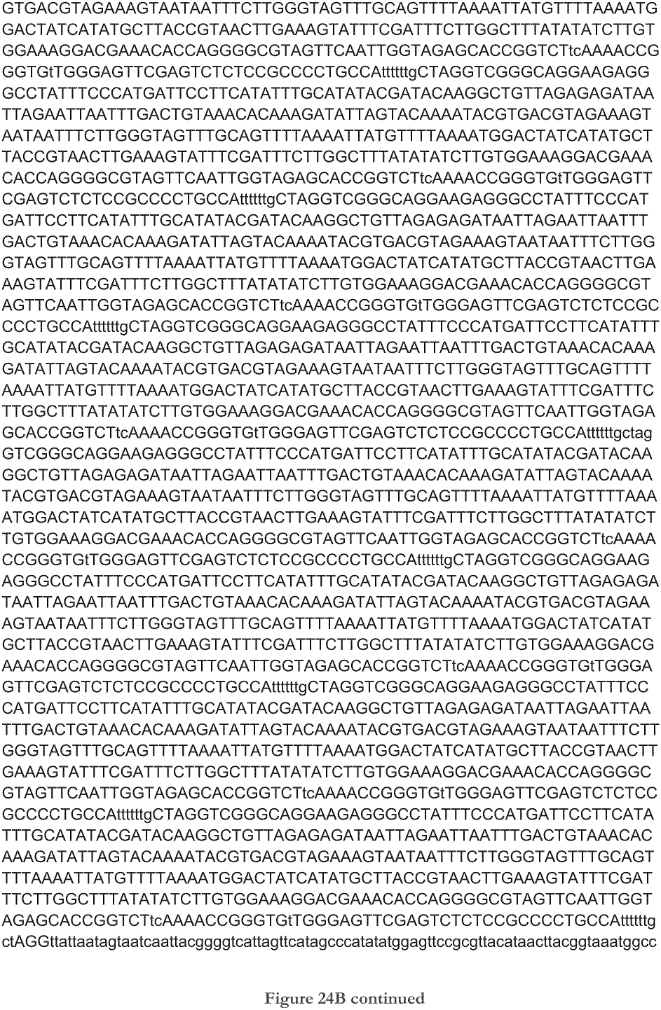

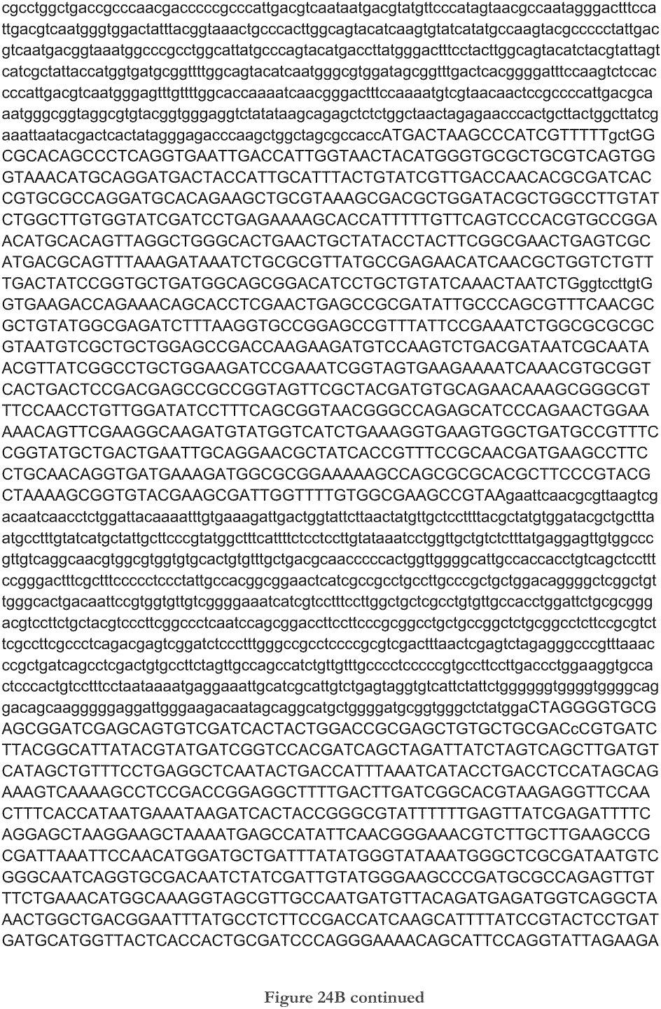

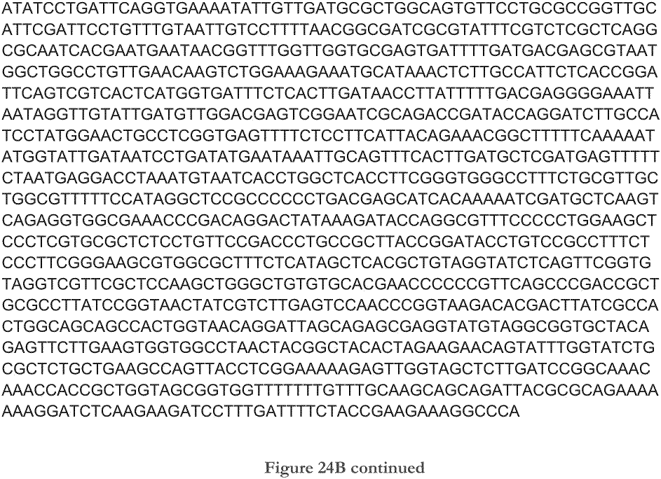

The plasmid maps and sequences used in the examples described below are found in FIGS. 17-24.

### Example 1: Packaging of AAV2

FIG. 3 shows packaging of AAV2 in HEK293T cells using constructs described in FIG. 2 to comparable yields relative to the original system. The qPCR titer of the original (WT) AAV2 is shown and the titer for the rest are shown relative to the WT. ΔVP1 and ΔVP2 represents AAV2 packaged using Cap genes from which the expression of VP1 and VP2, respectively, were eliminated. The virus still assembles efficiently in the absence of the minor capsid proteins. ΔVP1-CMV-VP1 and ΔVP2-CMV-VP2 represents AAV2 packaged using Cap genes from which the expression of VP1 and VP2, respectively, were eliminated and the respective proteins were expressed in trans from a CMV promoter. In these systems, the T454 residue in VP1 or VP2 were mutated to TAG and suppressed using a pyrrolysyl-tRNA synthetase/tRNA pair to incorporate an UAA(AzK), which are represented by ΔVP1-CMV-VP1-454AzK and ΔVP2-CMV-VP2-454AzK, respectively. Packaging yield of the virus in the presence or the absence of the UAA added to the media is shown.

### Example 2: Infectivity of the Packaged Virus

FIG. 4 shows the infectivity of the packaged, tittered viruses produced in FIG. 3 at constant titer measured by their ability to deliver and express an EGFP reporter gene in HEK293T cells. While ΔVP1 and ΔVP2 viruses package well, these show significantly attenuated infectivity. Supplying VP1 and VP2 in trans ΔVP1-CMV-VP1and ΔVP2-CMV-VP2) rescues the infectivity. ΔVP1-CMV-VP1-454AzK and ΔVP2-CMV-VP2-454AzK, viruses produced in the presence of the UAA show robust infectivity, while those in the absence does not. It should be noted that in the absence of UAA, the TAG mutants of the minor capsid proteins will fail to express.

### Example 3: Selective Fluorophore Labeling of Mutated AAV

FIG. 5 shows selective unnatural amino acid mutagenesis of individual minor capsid proteins in AAV capsid and their use to chemically attach a fluorophore. Following purification, different recombinant AAV2 preparations were labeled with a cyclooctynefluorophore, which selectively labels the azide group present in the UAA AzK. The top panel shows SDS-PAGE analysis of the AAV2 preparations, the bottom panel shows the fluorescence image of the same gel. As expected, the wild-type AAV2 does not show labeling, T454AzK (AzK in all 60 capsid proteins) show labeling of all the capsid proteins, while ΔVP1-CMV-VP1-454AzK and ΔVP2-CMV-VP2-454AzK show selective labeling of VP1 and VP2, respectively, thus demonstrating our ability to selectively label distinct minor capsid proteins.

### Example 4: Retargeting Efficiency of Mutated AAV

FIG. 6 shows the number of retargeting ligands attached to the AAV capsid dramatically affects its retargeting efficiency. Attaching cRGD ligands onto detargeted AAV2 capsids (where binding of the natural primary receptor, heparin sulfate proteoglycan receptor HSPG, was ablated by mutating key residues R588 and R587 to Ala; designated by amino acid residue location of VP1) enables it to selectively bind and infect cancer cell-lines such as SK-OV-3 that overexpress the αVβ3 integrin receptor. These graphs show the infectivity of the shown detargeted AAV2 mutants as they were incubated with cyclooctyne-cRGD over time, as it progressively functionalizes the AzK side chains with cRGD. For 454AA (60 AzK per capsid), the infectivity toward SK-OV-3 cells first go up and then come back down, suggesting an optimal number of cRGD per capsid needed for efficient retargeting. Over-modification of the capsid at later times leads to a loss of infectivity, likely by perturbing viral entry processes. For VP1-454AA and VP2-454AA (5 AzK each per capsid), infectivity goes up and reaches a plateau upon prolonged incubation, suggesting that attachment of 5 cRGD per capsid is not detrimental to AAV2 infectivity. However, the maximal infectivity reached for these mutants are low, suggesting that 5 cRGDs per capsid may not be sufficient for efficient retargeting. VP1+2-454AA (10 AzK per capsid) behaved just like VP1-454AA and VP2-454AA, but the infectivity upon prolonged incubation reaches levels similar to the optimal infectivity observed with T454-AA at the optimal degree of modification.

### Example 5: Precise Labeling of Engineered Cysteine Residues of AAV

FIGS. 7A-7C show precise labeling of AAV at engineered cysteine residues. (A) Packaging efficiency (qPCR) of AAV produced using wild-type Cap (WT), T454C mutant of Cap (T454C at all three capsid proteins), VP1-T454C (T454C mutant of the trans-substituted VP1), VP2-T454C (T454C mutant of the trans-substituted VP2). (B) Infectivity of these viruses measured by their ability to deliver and express an EGFP gene in HEK293T cells (FACS titer). (C) Selective labeling of the engineered 454-cysteine residue on VP1 by fluorescein-maleimide on VP1-454C virus. FAM-fluorescence image shows the result of the labeling reaction on WT and VP1-454C virus, whereas SYPRO stains all proteins.

### Example 6: Selective Incorporation of the UAA Into Either VP1 or VP2 or VP1+VP2

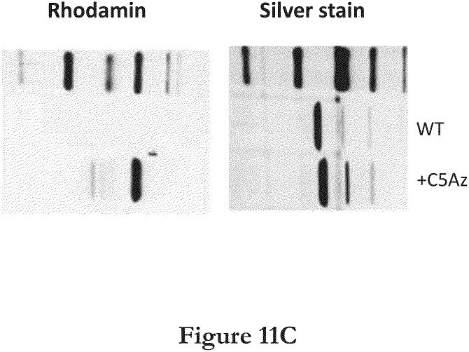

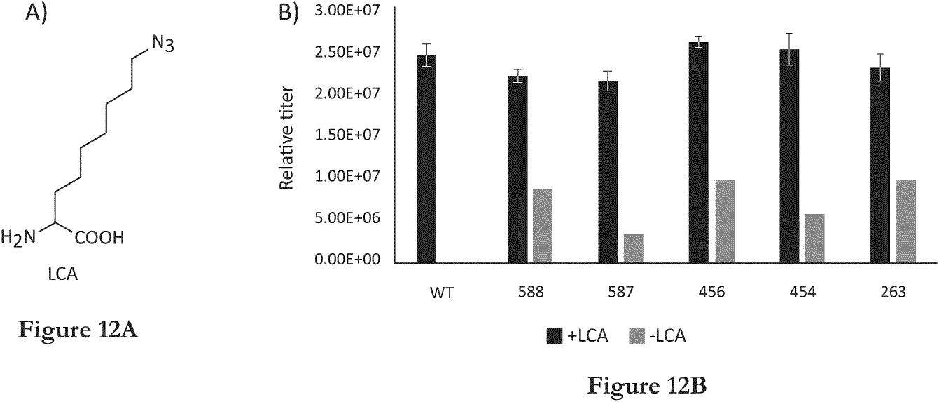

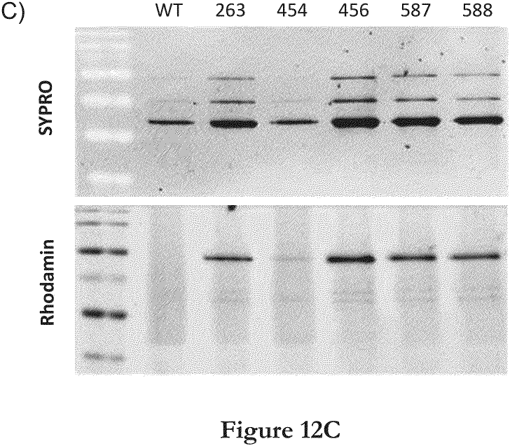

As shown in FIGS. 11A-C, C5Az was incorporated into either VP1 or VP2 or VP1 +VP2 by co-transfecting the HEK293T cells with a plasmid (pIDTsmart-ITR-GFP-4xEcLtR-LeuRS) encoding an engineered E. coli leucyl-tRNA synthetase (EcLeuRS) and tRNA pair that charge C5Az, as well as plasmids encoding [RC2-VPI-del + CMV-VP1-delVP23] or [RC2-VP2-del + CMV-VP2-delVP3] or [RC2-VP12-del + CMV-VP1-VP2-delVP3], respectively. The 454 position (VP1 numbering) in the desired protein was replaced with a TAG stop codon. A) Structure of C5Az, B) qPCR titers of the various preparations shown relative to wild-type AAV2 production, in the presence or absence of C5Az. C) Selective fluorescent labeling of VP1, using DBCO-rhodamine, on AAV2-VP1-454-C5Az.

### Example 7: Selective Incorporation of CpK Into VP1

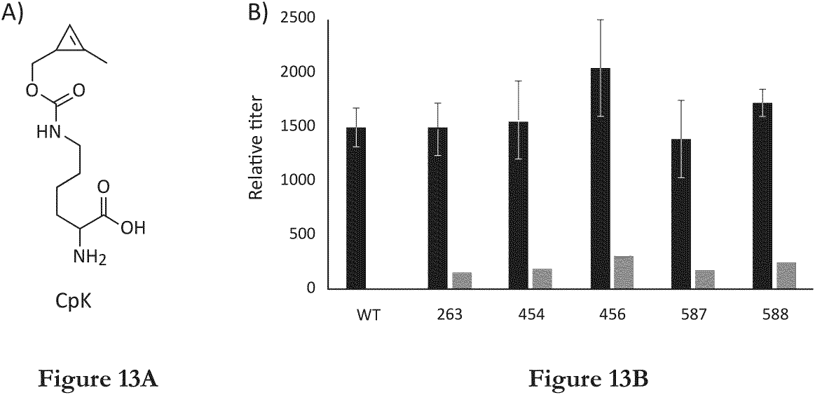

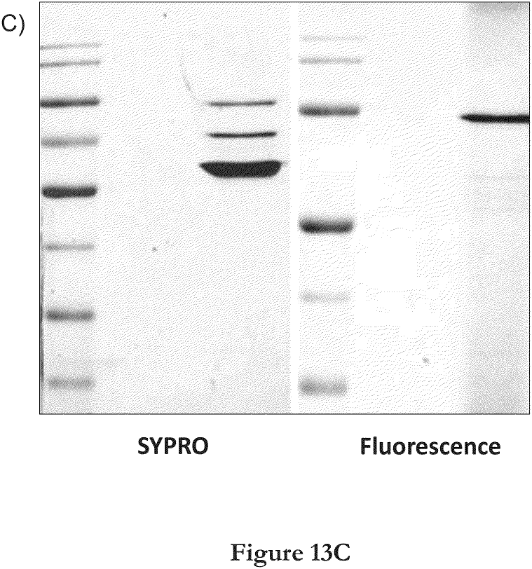

As shown in FIG. 13 A-C, CpK. was incorporated into VP1 by co-transfecting the HEK293T cells with a plasmid (pIDTsmart-ITR-GFP-4xEcLtR-LeuRS) encoding an engineered E. coli leucyl-tRNA synthetase (EcLeuRS) and tRNA pair that charge CpK, as well as plasmids encoding [RC2-VP1-del + CMV-VPl-delVJ>23]. Various sites (as indicated; VP1 numbering) in VP1 was replaced with a TAG stop codon. A) Structure of CpK, B) Relative titers of the various preparations shown relative to wild-type AAV2 production, in the presence or absence of LCA. C) Selective fluorescent labeling of VP1, using tetrazine-FITC, on AAV preparations incorporating LCA into site 456 of VP1.

### Example 8: Selective Incorporation of 5-HTP Into VP1

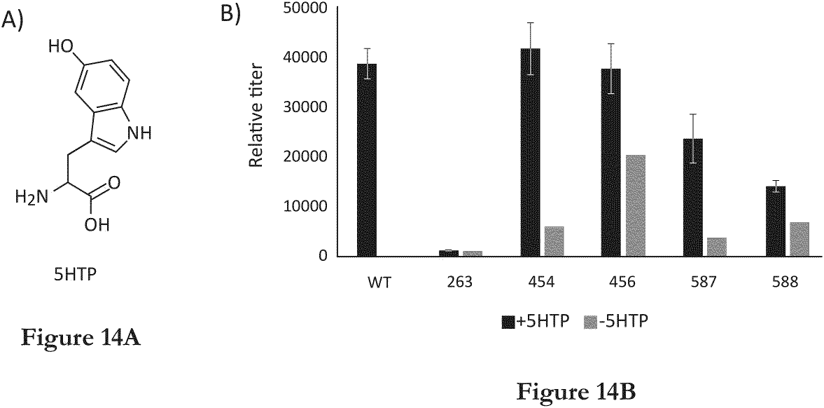

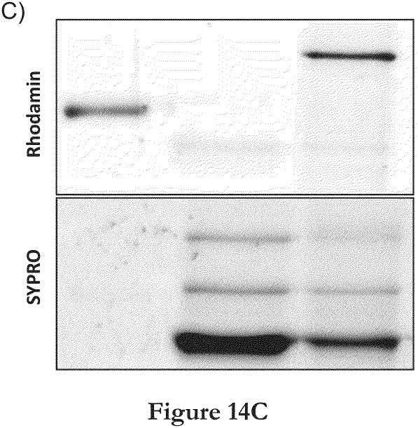

As shown in FIGS. 14A-C, 5HTP was incorporated into VP1 by co-transfecting the HEK293T cells with a plasmid (pIDTsmart-TrpRS-8xWtR-ITR-GFP) encoding an engineered E. coli tryptophanyl-tRNA synthetase (EcTrpRS) and tRNA pair that charge 5HTP, as well as plasmids encoding [RC2-VP1-del + CMV-VP1-delVP23]. Various sites (as indicated; VP1 numbering) in VP1 was replaced with a TGA stop codon. A) Structure of 5HTP, B) Relative titers of the various preparations shown relative to wild-type AAV2 production, in the presence or absence of 5HTP. C) Selective fluorescent labeling of VP1, using fluorescein amine and ferricyanide, on AAV preparations incorporating 5HTP into site 454 of VP1.

### Example 9: Incorporation UAAs Into VPI

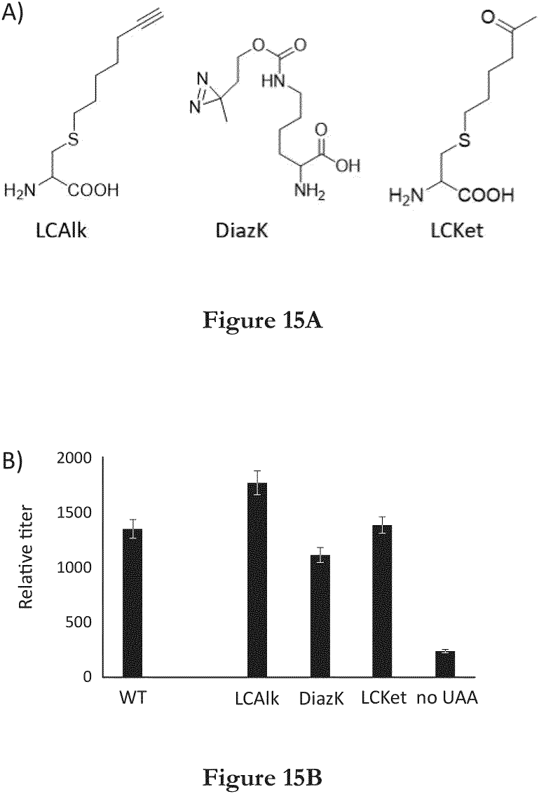

As shown in FIGS. 15A-B, sseveral other unnatural amino acids were incorporated into VP1 by co-transfecting the HEK293T cells with a plasmid (pIDTsmart-ITR-GFP-4xEcLtR-LeuRS) encoding an engineered E. coli leticyl-tRNA synthetase (EcLeuRS) and tRNA pair that charge these unnatural amino acids, as well as plasmids encoding [RC2-VP1-del + CMV-VP1-delVP23]. Site 454 (VP1 numbering) in VP1 was replaced with a TAG stop codon. A) Structure of the unnatural amino acids, B) Relative titers of the various mutant AAV2 preparations shown relative to wild-type AAV2 production, in the presence or absence of the indicated unnatural amino acids.

### Example 10: Modification of the LCA Site of AAV2-VP1-454-LCA

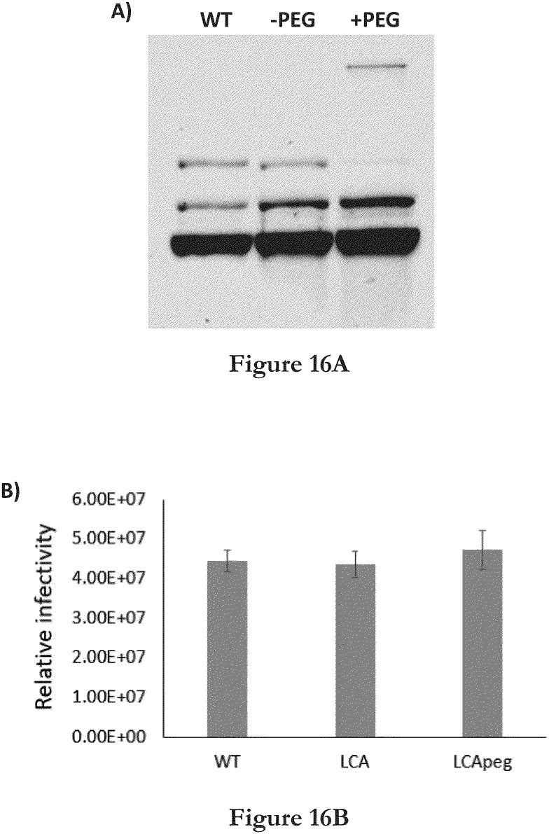

As shown in FIGS. 16A-B, AAV2-VP1-454-LCA, was selectively modified at the LCA site with 20 kDa polyethylene glycol (PEG) polymer using the corresponding PEG-DBCO conjugate. SDS-PAGE analysis shows selective labeling of VP1. Wild-type AAV2, AAV2-VP1-454-LCA, and PEG-modified AAV2-VP1-454-LCA show similar infectivity. Equal genome copies of each virus were added too HEK293 cells and the expression of the encoded luciferase reporter was monitored by a standard luciferase assay.

While this invention has been particularly shown and described with references to preferred embodiments thereof, it will be understood by those skilled in the art that various changes in form and details may be made therein without departing from the scope of the invention encompassed by the appended claims.

1. A genetically-modified adeno-associated virus (AAV) wherein the AAV capsid comprises at least one variant minor capsid protein VP1, VP2, or both VP1 and VP2, wherein the variant minor capsid protein is mutated at one, or more, amino acid residue sites to incorporate a natural amino acid or an unnatural amino acid (UAA) relative to the wild-type AAV VP1 or VP2 capsid protein.

2. The genetically-modified adeno-associated virus (AAV) of claim 1, comprising a variant minor capsid protein, wherein the variant capsid coding sequence is mutated at the translation origin of the AAV VP1, or VP2, or VP3 capsid protein open reading frames (ORF) to prevent translation of VP1, VP2 or both VP1 and VP2.

3. The genetically-modified AAV of claim 2, wherein the AAV capsid protein comprises SEQ ID NO:1, or a sequence comprising at least about 80% sequence identity of SEQ ID NO:1.

4. The genetically-modified AAV of claim 3, wherein a stop codon incorporated in the capsid protein(s) and the stop codon is a TAG, TAA or TGA codon.

5. The genetically-modified AAV of claim 1, comprising a variant minor capsid protein, wherein the natural amino acid residue is either a cysteine or a selenocysteine.

6. The genetically-modified AAV of claim 1, comprising a variant minor capsid protein, wherein the unnaturally-occurring amino acid is selected from the group consisting of: phenylalanine analogs; tyrosyl analogs; tryptophanyl analogs; or lysyl analogs.

7. The genetically-modified AAV of claim 6, wherein the analog comprises a formula selected from the group: 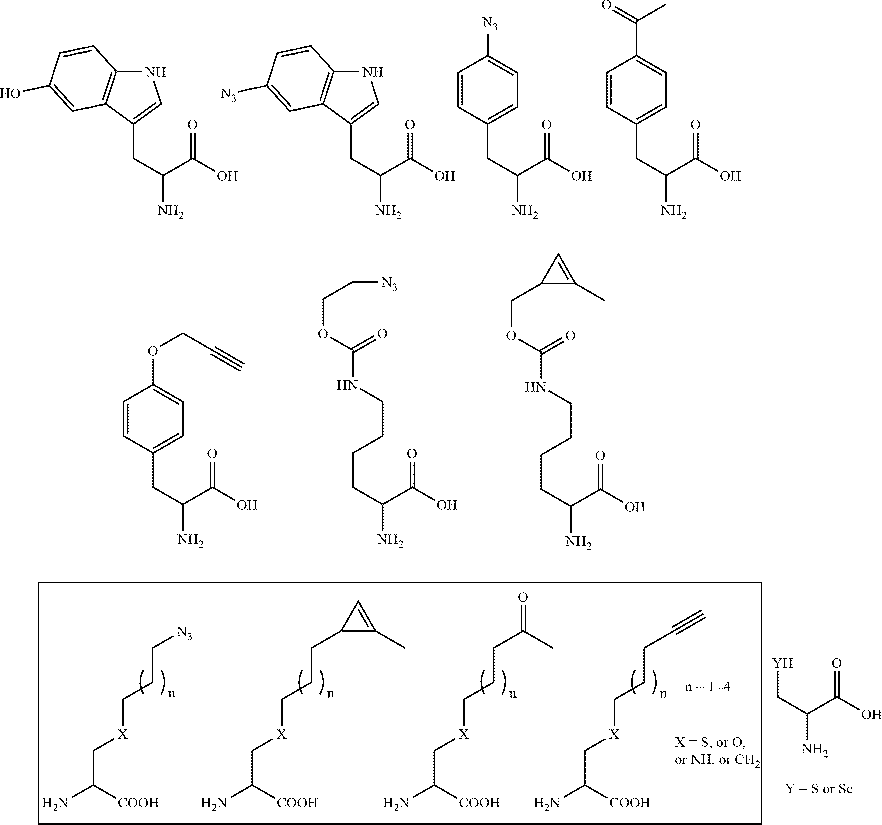
.

8. The genetically-modified AAV of claim 7, comprising a variant minor capsid protein, wherein the analogs are selected from the group consisting of: p-benzoylphenylalanine (pBpA); O-methyltyrosine (OMeY); 5-azidotryptophan; 5-propargyloxytryptopha; 5-aminotryptophan; 5-methoxytryptophan; 5-O-allyltryptophan; 5-bromotryptophan; azido-lysine (AzK); C5Az; LCA; Nε-acetyllysine (AcK); cyclopropene amino acid, N^{ε}-(1-methylcycloprop-2-enecarboxamido)-lysine (CpK); 5-hydroxy-tryptophan (5-HTP);LCAlk; DiaazK and LCKet.

9. The genetically-modified AAV of claim 1, wherein the natural or unnatural amino acid residue of the variant minor capsid protein incorporates a bioconjugation handle.

10. The genetically-modified AAV of claim 9, wherein the AAV capsid comprises 5 to 10 bioconjugation handles per capsid.

11. The genetically-modified AAV of claim 1 , comprising at least one variant minor capsid protein VP1, VP2 or both VP1 and VP2, wherein the AAV is characterized by:
a) infectivity of target cells comparable to wild-type AAV;
b) is packaged with titers comparable to wild-type AAV; or
c) both characteristics a) and b).

12. A genetically-modified infectious adeno-associated virus (AAV) wherein the genetically-modified AAV comprises a variant minor capsid protein VP1, VP2 or both VP1 and VP2 wherein VP1, VP2 or both VP1 and VP2 comprise one, or more mutated amino acid residues.

13. The genetically-modified infectious AAV of claim 12, wherein the variant capsid protein comprises one, or more mutated amino acid residues of VP1, VP2 or both VP1 and VP2 and the mutated amino acid residue site incorporates an unnatural amino acid (UAA).

14. The genetically-modified infectious AAV of claim 13, comprising a variant capsid protein, wherein the unnaturally-occurring amino acid is selected from the group consisting of: phenylalanine analogs; tyrosyl analogs; tryptophanyl analogs; or lysyl analogs.

15. The genetically-modified infectious AAV of claim 14, comprising a variant capsid protein, wherein the analogs are selected from the group consisting of: 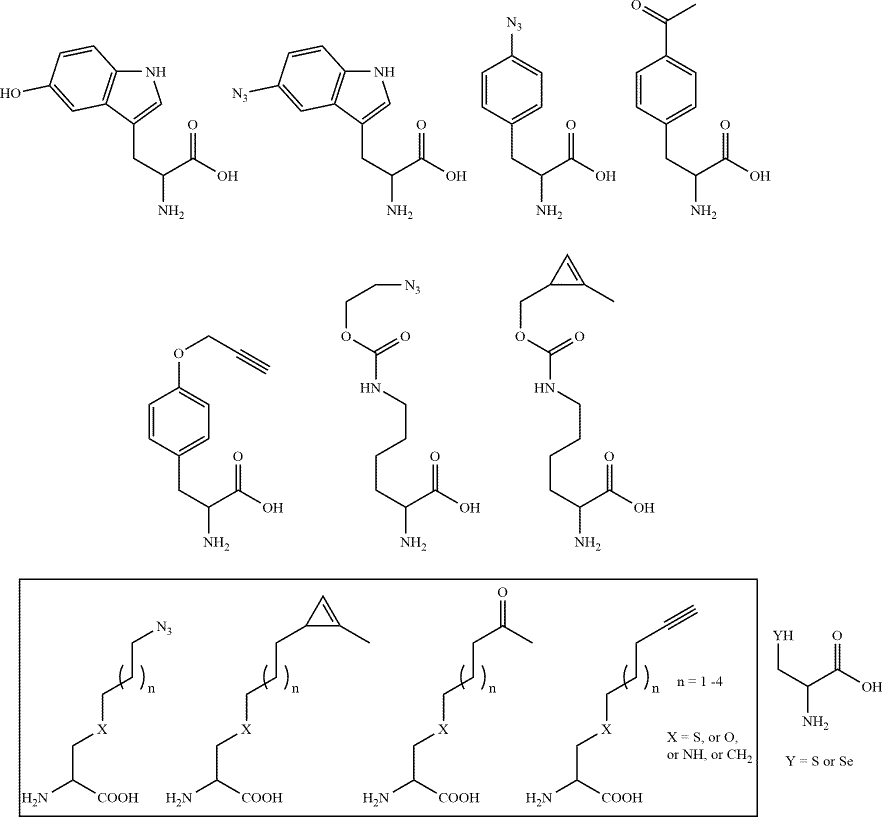
.

16. The genetically-modified infectious AAV of claim 12, wherein the variant capsid protein is VP1 comprising SEQ ID NO:1, or a sequence comprising at least about 80% sequence identity of SEQ ID NO:1.

17. The genetically-modified infectious AAV of claim 16, wherein the variant VP1 capsid protein is mutated at one, or more locations of the protein at position(s) 263, 454, 456, 587 and 588.

18. The genetically-modified infectious AAV of claim 12, wherein the variant capsid protein comprises one, or more mutated amino acid residues of VP1, VP2 or both VP1 and VP2 and the mutated amino acid residue site incorporates a naturally-occurring amino acid.

19. The variant capsid protein of claim 18, wherein the naturally-occurring amino acid is cysteine or selenocysteine.

20. The genetically-modified AAV of claim 18, wherein the mutated VP1 amino acid sequence comprises SEQ ID NO:1, or a sequence with at least about 80% sequence identity with Seq ID NO:1, wherein the variant VP1 capsid protein is mutated at one, or more locations at position(s) 263, 454, 456, 587 or 588.

21. The genetically-modified infectious AAV of claim 12, comprising the variant capsid protein comprising a mutated amino acid residue, wherein the mutated amino acid is conjugated with a chemical or biological (protein, nucleic acid, lipid, or carbohydrate) entity.

22. The genetically modified infectious AAV comprising a chemical or protein entity of claim 21, wherein the entity is selected from the group consisting of probes, small molecule ligands, peptides, cyclic peptides, nucleotides, polymers, or protein conjugates.

23. The genetically-modified infectious AAV comprising a chemical or protein entity of claim 22, wherein the entity is a cyclic peptide cRGD or polyethylene glycol.

24. A method of producing an infectious genetically-modified AAV, wherein the AAV comprises a variant AAV capsid protein, wherein VP1, VP2 or both VP1 and VP2 comprise one, or more mutated amino acid residue sites, relative to wild-type VP1, VP2 or both VP1 and VP2, the method comprising:
a) providing competent host cells in culture;
b) transfecting the cultured cells with one, or more plasmids comprising:
1) AAV variant VP3 that does not express VP1, or VP2, or both VP1 and VP2;
2) variant VP1, VP2 or both VP1 and VP2 with a suitable promoter that do not express VP3;
3) additional factors required for AAV expression;
c) providing the required unnatural amino acid; and plasmid encoding an engineered aminoacyl-tRNA synthetase/tRNA pair that selectively charge the unnatural amino acid in response to a stop codon;
d) culturing the cells under conditions sufficient for expression of the plasmid genes and assembly of the AAV;
thereby producing an infectious genetically-modified AAV comprising a variant AAV capsid protein, wherein VP1, VP2, VP3 or both VP1 and VP2 are mutated at one, or more amino acid residue sites relative to the wild-type AVV.

25-34. (canceled)

35. A therapeutic or antigenic composition comprising a and further genetically-modified AAV of claim 1 comprising one, or more therapeutic or antigenic gene constructs.

36. (canceled)

37. A method of treating a disease or condition in a subject, or of eliciting an immune response in a subject, the method comprising administering the therapeutic composition of claim 35 to the subject, wherein the composition comprises a gene construct encoding a protein or peptide in a therapeutic amount capable of decreasing or alleviating the disease or condition, or eliciting and immune response in the subject.

38. (canceled)

39. A kit comprising the genetically-modified AAV of claim 1.
